(19) United States
(12) Patent Application Publication
(10) Pub. No.: US 2002/0042645 A1
(43) Pub. Date: Apr. 11, 2002

(54) Drug eluting radially expandable tubular stented grafts

(76) Inventor: Donald T. Shannon, Trabuco Canyon, CA (US)

Correspondence Address: Edwards Lifesciences LLC, Law Dept., One Edwards Way, Irvine, CA, 92614, US

(21) Appl. No.: 09/997,829

(22) Filed: Nov. 29, 2001

Related U.S. Application Data
(63) Continuation-in-part of application No. 09/358,350, filed on Jul. 21, 1999.
Application No. 09/358,350 is a division of application No. 08/675,644, filed on Jul. 3, 1996.

(51) Int. Cl. A61F 2/06

(52) U.S. Cl. 623/1.13; 623/1.42; 623/1.49

(57) ABSTRACT

Drug eluting stented tubular grafts wherein the stent is coated with a coat comprising a composite of at least one biocompatible, pharmaceutically acceptable, bioerodible polymer and at least one therapeutic substance. The polymer may be a polyester. The therapeutic agent may include selective gene delivery vectors, sirolimus, actinomycin-D and paclitaxel. The stented grafts include an integrally stented embodiment and an internally stented embodiment. In each embodiment, the stent may be either self-expanding or pressure-expandable. Further, the stent may comprise a plurality of elements, wherein each said element comprises an undulating linear shape formed into a generally cylindrical configuration, and wherein each said element is connected to an adjacent neighbor element by at least one linear connector. A method for the treatment of cardiovascular disease by implantation of the stented graft, and an article of manufacture, comprising packaging material and the stented graft are also taught.

This is a continuation in part of application Ser. No. 09/358,350 filed Jul. 21, 1999, now pending, which is a division of U.S. Pat. No. 5,928,279, filed Jul. 3, 1996, issued Jul. 27, 1999.

### BACKGROUND ART

This invention pertains generally to medical devices and their methods of manufacture, and more particularly to drug eluting tubular grafts having radially expandable stents for implantation in a cavities or passageways (e.g., ducts or blood vessels) of the body, wherein the stents have polymer coats that possess the capability to release drugs.

A. Stents

The prior art includes a number of radially expandable stents which may be initially deployed in a radially collapsed state suitable for transluminal insertion via a delivery catheter, and subsequently transitioned to a radially expanded state whereby the stent will contact and engage the surrounding wall or the anatomical duct or body cavity within which the stent has been positioned. Such stents have been used to support and maintain the patency of blood vessel lumens (e.g., as an adjuvant to balloon angioplasty) and to structurally support and/or anchor other apparatus, such as a tubular endovascular grafts, at desired locations within a body cavity or passageway. For example, they may be used to anchor a tubular endovascular graft within a blood vessel such that the graft forms an internal conduit through an aneurysm or site of traumatic injury to the blood vessel wall.

Many stents of the prior art have been formed of individual member(s) such as wire, plastic, metal strips, or mesh that have been bent, woven, interlaced or otherwise fabricated into a generally cylindrical configuration. These stents of the prior art have generally been classified into two major categories: a) “self-expanding” stents, and b) “pressure expandable” stents. Some examples of stents of the prior art include those described in U.S. Pat. No. 5,405,377 (Cragg); U.S. Pat. No. 5,882,335 (Leone, et al.; U.S. Pat. No. 6,017,362 (Lau); U.S. Pat. No. 6,066,168 (Lau); U.S. Pat. No. 6,086,604 (Fischell et al.) and U.S. Pat. No. 6,117,165 (Becker).

i) Self-Expanding Stents

Self-expanding stents are typically formed of spring metal, shape memory alloy, or other material that is resiliently biased toward its fully radially expanded configuration or is otherwise capable of self-expanding to its fully radially expanded configuration without need for the exertion of outwardly directed radial force upon the stent by an extraneous expansion apparatus (e.g., a balloon or mechanical expander tool). Such self-expanding stents may be initially radially compressed and loaded into a small diameter delivery catheter or alternatively mounted upon the outer surface of a delivery catheter equipped with a means for restraining or maintaining the stent in its radially compressed state. Thereafter, the delivery catheter is inserted into the body and is advanced to a position wherein the stent is located at or near the site at which it is to be implanted. Thereafter, the stent is expelled from the delivery catheter and allowed to self-expand to its full radial diameter. Expansion of the stent causes the stent to frictionally engage the surrounding wall of the body cavity or passageway in which it has been positioned. The delivery catheter is then extracted, leaving the self-expanded stent at its intended site of implantation. Some examples of self-expanding stents of the prior art include those described in U.S. Pat. No. 4,655,771 (Wallsten et al.); U.S. Pat. No. 4,954,126 (Wallsten): U.S. Pat. No. 5,061,275 (Wallsten et al.); U.S. Pat. No. 4,580,568 (Gianturco); U.S. Pat. No. 4,830,003 (Wolf et al.); U.S. Pat. No. 5,035,706 (Gianturco et al.) and U.S. Pat. No. 5,330,400 (Song).

ii) Pressure-Expandable Stents

Pressure-expandable stents of the prior art are typically formed of metal wire, metal strips, or other malleable or plastically deformable material, fabricated into a generally cylindrical configuration. The pressure-expandable stent is initially disposed in a collapsed configuration having a diameter that is smaller than the desired final diameter of the stent when implanted in the blood vessel. The collapsed stent is first loaded into or mounted upon a small diameter delivery catheter. The delivery catheter is then advanced to its desired location within the vasculature, and a balloon or other stent-expansion apparatus (which may be formed integrally of or incorporated into the delivery catheter) is utilized to exert outward radial force on the stent, thereby radially expanding and plastically deforming the stent to its intended operative diameter whereby the stent frictionally engages the surrounding blood vessel wall. The material of the stent undergoes plastic deformation during the pressure-expansion process. Such plastic deformation of the stent material causes the stent to remain in its radially expanded operative configuration. The balloon or other expansion apparatus is then deflated/collapsed and is withdrawn from the body separately from, or as part of, the delivery catheter, leaving the pressure-expanded stent at its intended site of implantation.

Some examples of pressure-expandable stents of the prior art include those described in U.S. Pat. No. 5,135,536 (Hillstead); U.S. Pat. No. 5,161,547 (Tower); U.S. Pat. No. 5,292,331 (Boneau); U.S. Pat. No. 5,304,200 (Spaulding) and U.S. Pat. No. 4,733,665 (Palmaz).

iv. Drug Eluting Stents

In spite of the availability of the various stents of the prior art, a continuing need in the stented graft art is for a stented graft capable of providing drug therapy after implantation. The specific drug needed by patients who are being treated by the implantation of stented grafts varies with the type of pathology being treated—for example, whether cardiovascular, hepatic, or gastrointestinal. In the case of cardiovascular pathologies, it is pertinent that restenosis is observed in up to 50% of patients involved in angioplasty procedures. Restenosis refers to the reclosure of vessels by cellular or other invasion following vessel-clearing procedures. Restenosis is actually a natural healing process involving elements of the clotting cascade and later uncontrolled migration and proliferation of smooth muscle cells (SMC). The ultimate result is stenosis of the vessel—a return to the condition for which the treatment was initiated. Such cellular invasion is also a major problem in hepatic stenting procedures.

One of the original reasons for the use of stents in angioplasty was to minimize the impact of restenosis. Disappointingly, stents have been found not only to cause undesirable local thrombosis, but also to be ineffective in countering the effects of SMC migration and consequent restenosis. The consensus of medical opinion as of late 2001 is that it is unlikely that a single physiological process is responsible for restenosis, and thus it may be necessary to have different approaches for different clinical scenarios.

To address the restenosis problem, it has been proposed to provide therapeutic substances to the vascular wall. Although this could be done by means of systemic administration, for example orally or by injection, this route of administration subjects the patient to the general systemic effects of the drug. Such general systemic effects would include the possibility of systemic toxicity. By contrast, it has been proposed to administer the drugs locally by means of drug eluting stents. Here, ideally only the specific vasculature at issue would be affected by the action of the drug, whereas the general tissues of the patient would only be subjected to extremely small doses of the drug. Pertinent therapeutic substances include antiplatelet agents, anticoagulant agents, antimetabolic agents, vasoactive agents such as nitric oxide releasing agents, anti-inflammatory, antiproliferative, pro-endothelial, antisense and anti-migratory agents, all of which are embodied in the present invention. The administration of these agents, as well as antimicrobial agents to counter the possibility of infection is therefore of major interest in the stent and stented graft art.

Among further pharmacological agents that are of interest in the general connection discussed above is sirolimus, also known as rapamycin, an immunosuppressive and antiproliferative compound. Sirolimus is a macrocyclic lactone produced by Streptomyces hygroscopicus and has the molecular weight 914.2. Although early studies with sirolimus coated stents have been promising with regard to the reduction of restenosis, concerns remain in the medical community regarding drug dosing levels, the need for predictable drug deposition, and asymmetrical stent expansion that could lead to some spots getting a much higher concentration of drug than other spots. Furthermore, in the long-term, there is also the potential risk for stent malopposition, or that the pharmaceutical agent is merely delaying the effect of restenosis, that could eventually manifest itself. These issues will of course ultimately be examined by the use of suitable clinical tests.

Another drug of special interest in connection with stents is paclitaxel. Paclitaxel is a natural product that blocks vital mitotic cellular functions, and hence cellular proliferation. Paclitaxel has a molecular weight of 853.9. In a preliminary study, researchers at three German hospitals covered stents with low doses of paclitaxel designed to elute the drug for 28 days. During a six-month test period, no patient using a paclitaxel treated stent exhibited restenosis, whereas 11% of control patients exhibited restenosis.

One problem that has been associated with certain drug eluting stents is the development of an “edge effect” at the edges of the stents after placement. The “edge effect” comprises such phenomena as lumen reduction, neointimal proliferation inside the stented segment, plaque proliferation, and remodeling at the proximal and distal edges of the stent. By the use of the drug eluting radially expanded tubular stented grafts of the present invention the edge effect is drastically reduced or eliminated.

This invention generally embraces drug eluting stented grafts wherein the drug eluting capability is provided by a composite of drug material and a bioerodible polymer. A feature of the invention is the discovery of a particularly useful group of bioerodible polymers for this purpose. These polymers are fully described In U.S. Pat. No. 4,131,648 by Nam S. Choi and Jorge Heller, issued Dec. 26, 1978, assigned to Alza Corporation, and entitled “Structured Orthoester and Orthocarbonate Drug Delivery Devices”, which is incorporated herein in its entirety by reference. The patent discloses a class of polymers comprising a polymeric backbone having a repeating unit comprising hydrocarbon radicals and a symmetrical dioxycarbon unit with a multiplicity of organic groups bonded thereto. The polymers prepared by the invention have a controlled degree of hydrophobicity with a corresponding controlled degree of erosion in an aqueous or like environment to innocuous products. The polymers can be fabricated into coatings for releasing a beneficial agent, as the polymers erode at a controlled rate, and thus can be used as carriers for drugs for releasing drug at a controlled rate to a drug receptor, especially where bioerosion is desired.

v. Endovascular Brachytherapy Stents

A further approach to reduce restenosis after percutaneous coronary intervention is intravascular brachytherapy (VBT) which involves irradiation of the vasculature by an endovascular source such as a stent. Radiation sources for this purpose include palladium-103 (¹⁰³Pd), a low energy photon emitter. Other brachytherapy sources include ⁹²Ir, ³²P, and ¹⁸⁸Re. Sr/Y90 source trains have also been employed. The present invention provides a solution to the long-standing need for a stent for VBT.

vi. Gene Therapy

Recombinant Semliki Forest Virus (SFV) selectively transfers genes into cultured vascular smooth muscle cells leaving endothelial cells unaffected. Thus, SFV can function as a selective vector for balloon-injured vessels and can provide a pathway to deliver genes for the purpose of preventing restenosis. The administration of selective vectors such as SFV through stented graft delivery is therefore a further benefit of the present invention.

B. Elastomer Vascular Grafts

Elastomers, including fluoropolymers such as polytetrafluoroethylene, have been heretofore used for the manufacture of various types of prosthetic vascular grafts. These vascular grafts are typically of tubular configuration so as to be useable to replace an excised segment of blood vessel.

The tubular elastomer vascular grafts of the prior art have traditionally been implanted, by open surgical techniques, whereby a diseased or damaged segment of blood vessel is surgically excised and removed, and the tubular bioprosthetic graft is then anastomosed into the host blood vessel as a replacement for the previously removed segment thereof. Alternatively, such tubular prosthetic vascular grafts have also been used as bypass grafts wherein opposite ends of the graft are sutured to a host blood vessel so as to form a bypass conduit around a diseased, injured or occluded segment of the host vessel.

In general, many tubular prosthetic vascular grafts of the prior art have been formed of extruded, porous PTFE tubes. In some of the tubular grafts of the prior art, a PTFE tape is wrapped about and laminated to the outer surface of a tubular base graft to provide reinforcement and additional burst strength. Also, some of the prior tubular prosthetic vascular grafts have included external support member(s), such as a PTFE beading, bonded or laminated to the outer surface of the tubular graft to prevent the graft from becoming compressed or kinked during implantation. These externally supported tubular vascular grafts have proven to be particularly useful for replacing segments of blood vessel which pass through, or over, joints or other regions of the body which undergo frequent articulation or movement.

One commercially available, externally-supported, tubular vascular graft is formed of a PTFE tube having a PTFE filament helically wrapped around, and bonded to, the outer surface of the PTFE tube. (IMPRA Flex™ Graft, IMPRA, Inc., Tempe, Ariz.)

One other commercially available, eternally-supported, tubular vascular graft comprises a regular walled, PTFE tube which has PTFE reinforcement tape helically wrapped around, and bonded to, the outer surface of the PTFE tube and individual rings of Fluorinated Ethylene Propylene (FEP) rings disposed around, and bonded to, the outer surface of the reinforcement tape. (FEP ringed ePTFE vascular graft, W.L. Gore & Associates, Inc., Flagstaff, Ariz.)

C. Stented Grafts

The prior art has also included a number of “stented grafts”. These stented grafts typically comprise a self-expanding or pressure-expandable stent that is affixed to or formed within a pliable tubular graft. Because of their radial compressibility/expandability, these stented grafts are particularly useable in applications wherein it is desired to insert the graft into an anatomical passageway (e.g., blood vessel) while the graft is in a radially compact state, and to subsequently expand and anchor the graft to the surrounding wall of the anatomical passageway. More recently, methods have been developed for introducing and implanting tubular prosthetic vascular grafts within the lumen of a blood vessel, by percutaneous or minimal incision means. Such endovascular implantation initially involves transluminal delivery of the graft, in a compacted state, by way of a catheter or other transluminally advancable delivery apparatus. Thereafter, the graft is radially expanded and anchored to the surrounding blood vessel wall, thereby holding the graft at its intended site of implantation within the host blood vessel. An affixation apparatus such as a stent may be utilized to anchor at least the opposite ends of the tubular graft to the surrounding blood vessel wall. One particular application for endovascular grafts of this type is in the treatment of vascular aneurysms without requiring open surgical access and resection of the aneurysmic blood vessel. Also, such stented grafts may also be useable to treat occlusive vascular disease—especially in cases where the stented graft is constructed in such a manner that the tubular graft material forms a complete barrier between the stent and the blood that is flowing through the blood vessel. In this manner the tubular graft material may serve as a smooth, biologically compatible, inner “covering” for the stent, thereby preventing a) turbulent blood-flow as the blood flows over the wire members or other structural material of which the stent is formed, b) immunologic reaction to the metal or other material of which the stent is formed, and c) a barrier to separate a diseased or damaged segment of blood vessel from the blood-flow passing therethrough. Such prevention of turbulent blood-flow and/or immunologic reaction to the stent material is believed to be desirable as both of these phenomena are believed to be associated with thrombus formation and/or restenosis of the blood vessel. Other uses for stented grafts may include restoring patency to, or re-canalizing, other anatomical passageways such as ducts of the biliary tract, digestive tract and/or genitourinary tract.

A number of specific desiderata are of special importance with regard to the suitability of particular expandable stent designs for incorporation into a drug eluting stented graft. Among these are high flexibility, high hoop strength of the stent in its expanded form, minimal foreshortening of the stent in the course of its transition from a compressed state to an expanded state, and minimal “dog bone effect.” High flexibility is necessary in order for the drug eluting stented graft to be smoothly inserted into regions of convolution. High hoop strength is necessary in order that the stent will fulfill its primary function of holding a lumen open. Minimal foreshortening is necessary to avoid excessive puckering, wrinkling or invagination of the elastomer graft material during expansion of the stent from its compressed state to its expanded state. The “dog bone effect” is the tendency of the ends of a stent to expand before the middle portion expands. This results in a “bone-shaped” structure in which the ends of the stent have expanded more than the middle portions. In addition to other undesirable characteristics of this expansion mode, excessive foreshortening accompanies “dog-boning.” Thus there remains a need for improved drug eluting stented grafts having high flexibility, high hoop strength of the stent in its expanded form, minimal foreshortening of the stent in the course of its transition from a compressed state to an expanded state, and minimal “dog bone effect.” Embodiments of this invention to solve some of the problems enumerated above have been the subject of a copending continuation-in-part filed very recently.

Variations on the known medical use of stented grafts adapted for drug elution have not been forthcoming, despite recent developments in the technology related to stent technology. Even though stented grafts are used extensively in medical practice, prior devices, products, or methods available to medical practitioners have not adequately addressed the need for advanced methods and apparatus for minimizing the deficiencies in drug elution as set forth above.

The present invention embraces and finally addresses the clear need for advanced methods and apparatus for solving the long-standing needs in drug eluting stents as set forth above. Thus, as pioneers and innovators attempt to make methods and apparatus for stented grafts cheaper, more universally used, and of higher quality, none has approached the desiderata outlined above in combination with simplicity and reliability of operation, until the teachings of the present invention. It is respectfully submitted that other references merely define the state of the art or show the type of systems that have been used to alternately address those issues ameliorated by the teachings of the present invention. Accordingly, further discussions of these references has been omitted at this time due to the fact that they are readily distinguishable from the instant teachings to one of skill in the art.

### OBJECTS AND SUMMARY OF THE INVENTION

It is an object of the present invention to provide a drug eluting stented graft of high flexibility. It is another object of the present invention to provide a drug eluting stented graft of high hoop strength of the stent in its expanded form. It is still another object of the present invention to provide a drug eluting stented graft having minimal foreshortening of the stent in the course of its transition from a compressed state to an expanded state. It is yet still another object of the present invention to provide a drug eluting stented graft having minimal “dog bone effect” in the course of its transition from a compressed state to an expanded state. It is even yet still another object of the present invention to provide a drug eluting stented graft having minimal puckering, wrinkling or invagination of the elastomer graft material during expansion of the stent from its compressed state to its expanded state. It is a further object of the present invention to provide a drug eluting stented graft that can be smoothly inserted into regions of convolution. It is yet a further object of the present invention to provide a means to administer drugs locally by means of drug eluting stented grafts. It is yet still a further object of the present invention to administer antiplatelet agents, anticoagulant agents, antimetabolic agents, vasoactive agents such as nitric oxide releasing agents, anti-inflammatory, antiproliferative, pro-endothelial, anti-migratory agents, and antimicrobial agents by means of drug eluting stented grafts. It is even still a further object of the present invention to provide a drug eluting stented graft that can provide sirolimus or paclitaxil to a local area. It is even yet still a further object of the present invention to provide a drug eluting stented graft whereby drug delivery is regulated both by a drug delivery coating on a stent and the porosity of the polymer comprising the stented graft.

These and other objects are accomplished by the parts, constructions, arrangements, combinations and subcombinations comprising the present invention, the nature of which is set forth in the following general statement, and preferred embodiments of which—illustrative of the best modes in which applicant has contemplated applying the principles—are set forth in the following description and illustrated in the accompanying drawings, and are particularly and distinctly pointed out and set forth in the appended claims forming a part hereof.

The present invention is directed to improved tubular drug eluting stented grafts and their methods of manufacture. The present invention may exist in numerous embodiments, including those wherein the stent component of the graft is formed integrally within the tubular graft or wherein it is situated on the inner surface of the tubular graft. Embodiments of the invention may be self-expanding, incorporating a self-expanding stent, or pressure-expandable, incorporating a pressure-expandable stent.

In accordance with one embodiment of the invention, there is provided an improved integrally drug eluting stented elastomer graft which comprises a tubular base graft, a radially expandable stent surrounding the outer surface of the tubular base graft, and an outer elastomer layer. The tubular outer layer is fused to the tubular base graft through lateral openings or perforations formed in the stent. A drug delivery coating is disposed on the stent.

In accordance with another embodiment of the invention, there is provided an improved internally drug eluting stented, tubular elastomer graft which comprises a radially compressible/expandable stent having a elastomer tube coaxially disposed outside of the stent, with the inner surface of the tubular elastomer graft being fused or attached to the stent. A drug delivery coating is applied to or formed on the stent.

The invention may be manufactured by a method which comprises the steps of: a) initially positioning a generally cylindrical stent of either the self-expanding or pressure-expandable variety in contacting coaxial relation with the tubular base graft and/or the tubular outer layer, upon a cylindrical mandrel or other suitable support surface, and b) subsequently fusing (i.e., heating to a lamination temperature) the assembled components (i.e., the stent in combination with the inner base graft and/or outer tubular layer) of the drug eluting stented graft into a unitary drug eluting stented graft structure. Heating is accomplished using a “waffle-iron” heater wherein heat is applied only to areas that correspond to the spaces not occupied by the stent. The purpose of the “waffle-iron” heater is to avoid heating the drug covering the stent to its decomposition temperature. Such heating will cause the outer layer to heat fuse to the inner base graft through the openings that exist in the stent. An alternative to the “waffle-iron” heater is to use a laser beam controlled by a computer to “hit” only the areas corresponding to the openings that exist in the stent. Computer controlled laser beams to accomplish such a purpose are known in the art. In integrally drug eluting stented embodiments where both the tubular base graft and the tubular outer layer are present, such heating will additionally cause the tubular outer layer to fuse to the inner tubular base graft, through lateral openings or perforations which exist in the stent.

By the above-described materials and methods of construction, the drug eluting stented elastomer grafts of the present invention are capable of radially expanding and contracting without excessive puckering, wrinkling or invagination of the graft material. Furthermore, in embodiments wherein the stent is constructed of individual members which move or reposition relative to one another during respective expansion and contraction of the drug eluting stented graft, the manufacturing methods and materials of the present invention render the elastomer sufficiently strong and sufficiently firmly laminated or fused so as to permit such relative movement of the individual members of the stent without tearing or rupturing of the tubular graft.

Further objects and advantages of the invention will become apparent to those skilled in the art upon reading and understanding the following detailed description and the accompanying drawings.

### BRIEF EXPLANATION OF THE DRAWINGS

FIG. 1 is a perspective view of a drug eluting radially expandable tubular stented graft of the present invention, wherein a portion of the graft has been inserted into a tubular catheter.

FIG. 1a is an enlarged perspective view of a segment of FIG. 1.

FIG. 2 is an enlarged, cut-away, elevational view of a drug eluting radially expandable tubular stented graft of the present invention.

FIG. 3a is an enlarged perspective view of a portion of the drug eluting radially expandable stent of the present invention incorporated in the graft of FIG. 2.

FIG. 3b is an enlarged cross-sectional view through line 3b-3d of FIG. 3a.

FIGS. 4a-4f are a step-by-step illustration of a preferred method for manufacturing a drug eluting radially expandable tubular stented graft of the present invention.

FIG. 5 is a schematic illustration of an alternative electron beam deposition method which is usable for depositing a coat comprising a composite of at least one polymer and at least one therapeutic substance on the drug eluting radially expandable stent of the present invention.

FIG. 6 is a schematic diagram of a “waffle iron” heating apparatus which is useable in the manufacture of a drug eluting radially expandable stent of the present invention.

FIG. 7 is a perspective view of a section of a drug eluting radially expandable stent of the present invention that illustrates portions of three elements each comprising an undulating zigzag shape.

FIG. 7a is an enlarged longitudinal sectional view of a drug eluting radially expandable stent of the invention shown in FIG. 7 taken along section line 7a therein.

FIG. 8 is a perspective view of a section of a drug eluting radially expandable stent of the invention that illustrates portions of two elements each comprising an undulating sinusoidal shape.

FIG. 8a is an enlarged longitudinal sectional view of a drug eluting radially expandable stent of the invention shown in FIG. 8 taken along section line 8a therein.

FIG. 9 is an enlarged, cut-away, elevational view of a drug eluting radially expandable tubular stented graft of the present invention.

### DETAILED DESCRIPTION OF THE PREFERRED EMBODIMENTS

The following detailed description is provided for the purpose of describing and illustrating presently preferred embodiments of the invention only, and is not intended to exhaustively describe all possible embodiments in which the invention may be practiced.

The drug delivery polymers in the drug delivery stents of the invention can be used as a single film or in a number of layers made of the same or of different polymers. They have a controlled degree of hydrophobicity in the environment of use and they erode into innocuous products at a continuous rate which exhibits no known deleterious effects on the environment or towards an animal body.

The term “hydrophobicity” as used above and in the remainder of the specification broadly refers to the property of the polymers not to absorb appreciable amounts of water. The terms “erodible” and “bioerodible” as used herein define the property of the polymers to break down as a unit structure or entity in a non-biological or in a biological environment over a period of time to innocuous products. The terms “erosion”, “bioerode” and “bioerosion” generally define the method and environment where breakdown or degradation of the polymer occurs. The phrase “prolonged period of time” as used herein, generally means the period between the start of erosion or the breakdown of the polymers when the polymers are placed in a moisture laden environment and that period in time when the polymer is gone. Depending upon the structure and dimensions of the stented graft, such as number of layers and thickness, the period may continue over days, several months such as ninety days, one hundred and eighty days, a year or longer. The environment includes aqueous and aqueous-like biological environments.

The term “therapeutic agent” as used in the specification and accompanying claims includes any compound, mixture of compounds, or composition of matter consisting of a compound and a carrier, which when released from a stented graft produces a beneficial and useful result. The drugs that may be administered include inorganic and organic drugs without limitation. The agents or drugs also can be in various forms, such as uncharged molecules, components of molecular complexes, pharmacologically acceptable salts such as hydrochloride, hydrobromide, sulfate, laurate, palmitate, phosphate, nitrate, borate, acetate, maleate, tartrate, oleate, and salicylate. For acidic drugs, salts of metals, amines, or organic cations, for example quaternary ammonium can be employed. Furthermore, simple derivatives of drugs such as esters, ethers, and amides that have solubility characteristics that are suitable for the purpose of the invention can be employed. Also, an agent or drug that is water insoluble can be used in a form that is a water soluble derivative thereof to effectively serve as a solute, and on its release from the device, is converted by enzymes, hydrolyzed by body pH, or metabolic processes to the original form or to a biologically active form. Additionally, agents or drug formulations within the devices can have various art known forms such as solutions, dispersions, pastes, particles, granules, emulsions, suspensions and powders.

The drug eluting stented grafts of the present invention utilize bioerodible, agent-release, rate controlling materials that bioerode at a controlled and continuous rate concurrently with the release of agent at a corresponding controlled and continuous rate. Devices made with the present bioerodible polymers are reliable and easy to use for releasing an agent as they normally require intervention or handling only at the time when the device is positioned in the patient. Additionally, the devices can be made to release an agent at a zero rate or at a variable rate by controlling the molecular weight and composition of the polymer, by controlling the concentration of the agent in the polymer and the surface area exposed, and by making the devices with different drug delivery polymers that undergo bioerosion and agent release at different rates, or by fabricating the polymer coated stents integrally into stented grafts wherein the graft polymer controls drug release.

The polymers comprising a carbon-oxygen backbone having a dioxycarbon moiety with a plurality of organic groups pendant from the dioxycarbon. The bioerodible polymers are represented by the following general formula:

[chemical structure]

WHEREIN R₁ is a di, tri or tetravalent alkylene, alkenylene, alkyleneoxy, cycloalkylene, cycloalkylene substituted with an alkyl, alkoxy or alkenyl, cycloalkenylene, cycloalkenylene substituted with an alkyl, alkoxy or alkenyl, arylene, or a arylene substituted with an alkyl, alkoxy or alkenyl, R₂ and R₃ are alkyl, alkenyl, alkoxy, alkenyloxy, alkylene, alkenylene, alkyleneoxy, alkenyleneoxy, alkylenedioxy, alkenylenedioxy, aryloxy, aralkyleneoxy, aralkenyleneoxy, aralkylenedioxy, aralkenylenedioxy, oxa, or OR₁O with R₁ defined as above; and wherein, (a) R₁ is divalent when R₂ and R₃ are alkyl, alkenyl, alkoxy, or alkenyloxy, with at least one of R₂ and R₃ an alkoxy or alkenyloxy; (b) R₁ is divalent when R₂ and R₃ are intramolecularly covalently bonded to each other and to the same dioxycarbon atom to form a heterocyclic ring or a heterocyclic ring substituted with an alkyl, alkoxy or alkenyl when R₂ is an alkyleneoxy or alkenyleneoxy and R₃ is an alkyleneoxy, alkenyleneoxy or alkylene; (c) R₁ is divalent when R₂ and R₃ are intramolecularly covalently bonded to each other and to the same dioxy carbon atom to form a fused polycyclic ring or a fused polycyclic ring substituted with an alkyl, alkoxy or alkenyl when R₂ is an oxa, alkyleneoxy or alkenyleneoxy and R₃ is aryloxy, aralkyleneoxy, aralkenyleneoxy or aralkylene; (d) R₁ is divalent when R₂ or R₃ is an OR₁O bridge between polymer backbones bonded through their dioxycarbon moieties, and the other R₂ or R₃ is an alkyl, alkenyl, alkyloxy, or alkenyloxy; (e) R₁ is tri or tetravalent when R₂ and R₃ are covalently bonded to each other and to the same dioxycarbon atom to form a heterocyclic ring or a heterocyclic ring substituted with an alkyl, alkoxy or alkenyl when R₂ is an alkyleneoxy or alkenyleneoxy and R₃ is an alkyleneoxy, alkenyleneoxy or alkylene; (f) R₁ is tri or tetravalent when R₂ and R₃ are covalently bonded to each other and to the same dioxy carbon atom to form a fused polycyclic ring or fused polycyclic ring substituted with an alkyl, alkoxy or alkenyl when R₂ is an oxa, alkyleneoxy or alkenyleneoxy and R₃ is aryloxy, aralkyleneoxy, aralkenyleneoxy or aralkylene.

The polymers include homopolymers, copolymers of the random and block types formed by reacting monomers or mixtures of preformed homopolymers and/or copolymers, branched polymers and cross-linked polymers. Thermoplastic linear polymers are afforded when R₁ is divalent, R₂ and R₃ are substituted with a noncross-linking group or are bonded intramolecularly; thermosetting cross-linked polymers are produced when R₁ is divalent and R₂ and R₃ is intermolecularly bonded between different polymeric backbones; and, thermosetting cross-linked polymers result when R₁ is tri or tetravalent and R₂ and R₃ are substituted with noncross-linking groups, or bonded intramolecularly.

A typical drug eluting radially expandable tubular stented graft having a stent coated with a polymer having an erosion rate of about 2μ per hour in a biological aqueous environment with a physiological pH of 6 to 8 and a drug concentration of 5% can be prepared as follows: To 2.375 g of poly(2,2-dioxo-trans-1,4-cyclohexane dimethylene tetrahydrofuran) was added 0.125 g of hydrocortisone and the ingredients heated to 150° C. to give a melt. The drug was dispersed throughout the melt by mixing the ingredients for 5 minutes to give a good dispersion. The mixing was performed in a dry, inert environment, at atmospheric pressure, and with dry equipment. A stent was dipped into the molten polymer and withdrawn in order to coat the stent. After cooling, the stent was fabricated into a stented graft. The graft, was placed in a biological aqueous environment where the coat bioeroded and released steroid for the potential management of inflammation.

A. The Structure of an Integrally Drug Eluting Stented PTFE Graft

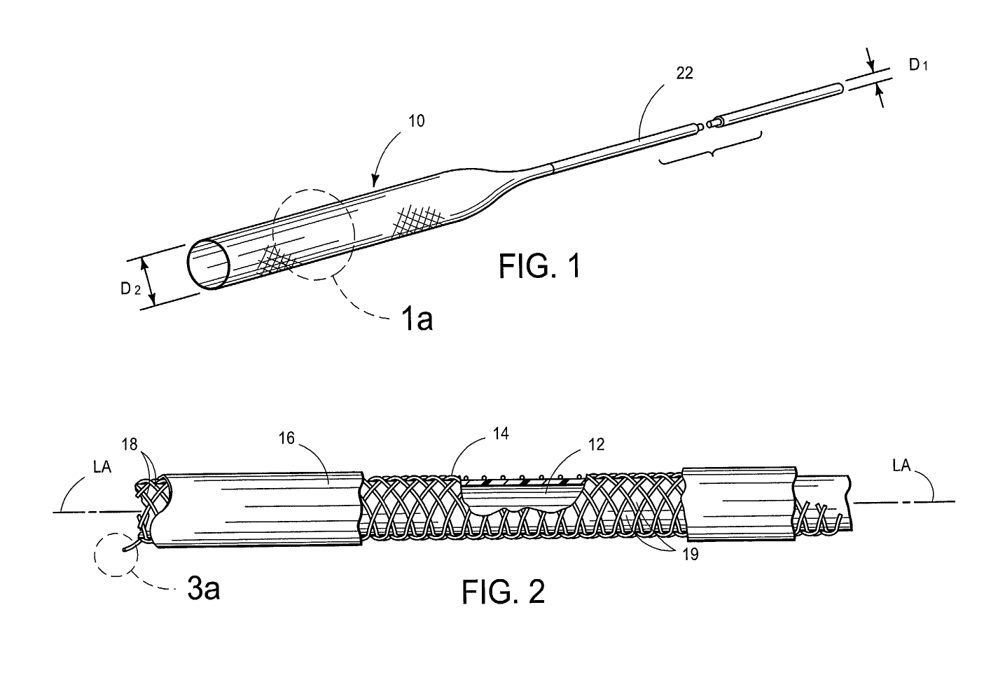

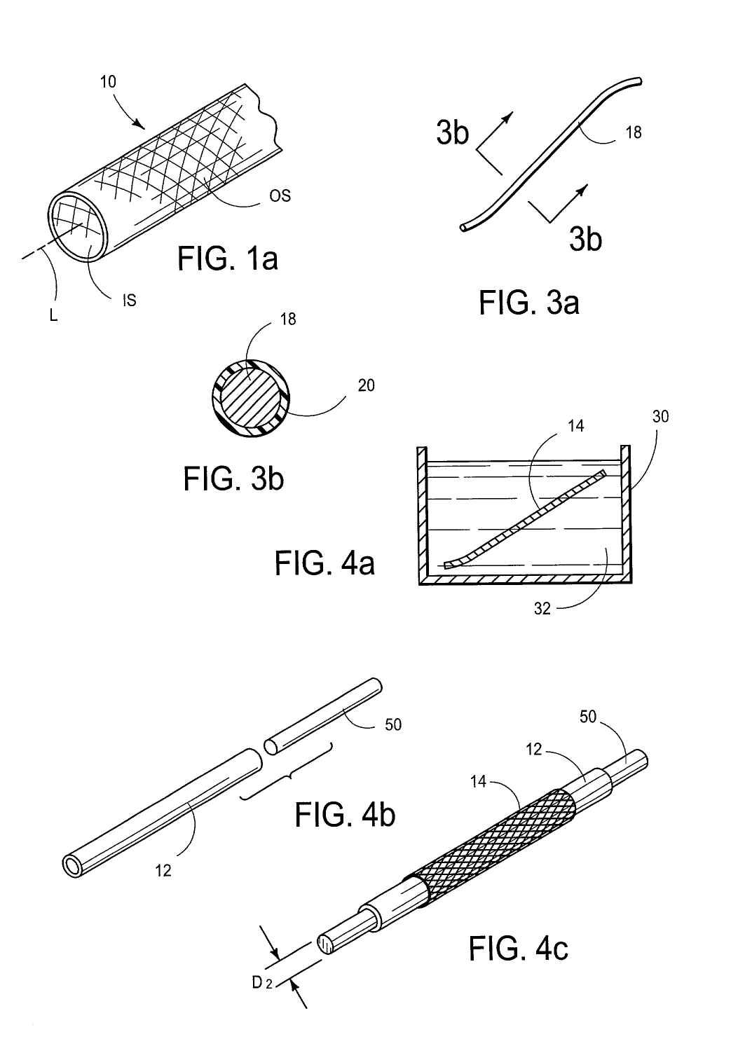

With reference to FIGS. 1-3b, there is shown a drug eluting radially expandable tubular stented graft 10 of the present invention. Graft 10 comprises a tubular base graft 12, a stent 14 coated with a coat comprising a composite of at least one polymer and at least one therapeutic substance, and an outer layer of elastomer 16. Stent 14 is formed of metal, such as an alloy of cobalt, chromium, nickel or molybdenum, wherein the alloying residue is iron. One specific example of a commercially available alloy which may is usable to form the wires 18 of the stent 14 is Elgiloy (The Elgiloy Company, 1565 Fleetwood Drive, Elgin, Ill. 60120. Stent 14 may be radially compressed to a smaller diameter D₁ and radial constraint, as may be applied by the surrounding wall of the tubular delivery catheter 22 shown in FIG. 1, may be applied to hold the stent 14 in such radially compressed state (diameter D₁). Thereafter, when the radial constraint is removed from the stent 14, the stent 14 will resiliently spring back to its radially expanded diameter D₂. Stent 14 may be a shape memory alloy that can alternately exist in a first and a second crystalline state, or it may be a pressure-expandable stent. Stent 14 may be formed of a metal alloy comprising at least two elements selected from the group consisting of iron, cobalt, chromium, nickel, titanium, niobium, and molybdenum. For example, the alloy may comprise at least about 51% to about 59% nickel and the remainder comprising titanium. Alternatively, it may comprise about 0.25% chromium, at least about 51% to about 59% nickel, and the remainder comprising titanium.

B. Preparation of the PTFE Tubular Base Graft

i.) Preparation of Paste

The manufacture of tubular base graft 12 begins with the step of preparing a PTFE paste dispersion for subsequent extrusion. This PTFE paste dispersion may be prepared by known methodology whereby a fine, virgin PTFE powder (e.g., F-104 or F-103 Virgin PTFE Fine Powder, Dakin America, 20 Olympic Drive, Orangebury, N.Y. 10962) is blended with a liquid lubricant, such as odorless mineral spirits (e.g., Isopar®, Exxon Chemical Company, Houston, Tex. 77253-3272), to form a PTFE paste of the desired consistency.

ii.) Extrusion of Tube

The PTFE-lubricant blend dispersion is subsequently passed through a tubular extrusion dye to form a tubular extrudate.

iii.) Drying

The wet tubular extrudate is then subjected to a drying step whereby the liquid lubricant is removed. This drying step may be accomplished at room temperature or by placing the wet tubular extrudate in an oven maintained at an elevated temperature at or near the lubricant's dry point for a sufficient period of time to result in evaporation of substantially all of the liquid lubricant.

iv.) Expansion

Thereafter, the dried tubular extrudate is longitudinally expanded or longitudinally drawn at a temperature less than 327° C. and typically in the range of 250-326° C. This longitudinal expansion of the extrudate may be accomplished through the use of known methodology, and may be implemented by the use of a batch expander. Typically, the tubular extrudate is longitudinally expanded by an expansion ratio of more than two to one (2:1) (i.e., at least two (2) times its original length).

v.) Sintering

After the longitudinal expansion step has been completed, the expanded PTFE tube is subjected to a sintering step whereby it is heated to a temperature above the sintering temperature of PTFE (i.e., 350-370° C.) to effect amorphous-locking of the PTFE polymer. The methodology used to effect the sintering step, and the devices used to implement such methodology, are known in the art. The PTFE tape 16 may be manufactured by any suitable method, including the general method for manufacturing expanded PTFE tape.

C. Coating of Stent 14

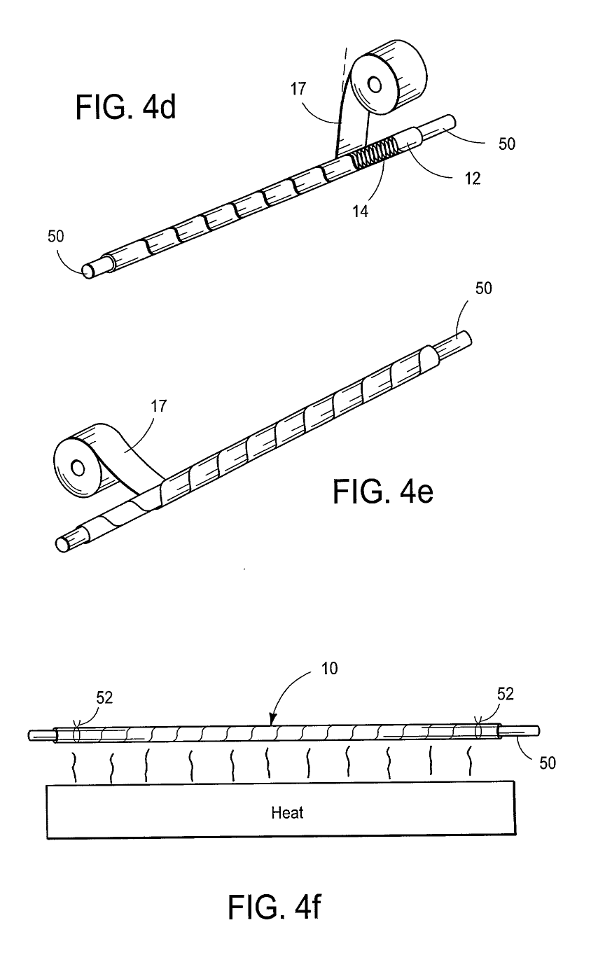

Prior to assembly of the components of graft 10, stent 14 is coated with a coating 20 comprising a composite of at least one polymer and at least one therapeutic substance. For example, it may be coated with a polymer having an erosion rate of about 2μ per hour in a biological aqueous environment with a physiological pH of 6 to 8 and a drug concentration of 5% prepared as follows: To 2.375 g of poly(2,2-dioxo-trans-1,4-cyclohexane dimethylene tetrahydrofuran) was added 0.125 g of hydrocortisone and the ingredients heated to 150° C. to give a melt. The drug was dispersed throughout the melt by mixing the ingredients for 5 minutes to give a good dispersion. The mixing was performed in a dry, inert environment, at atmospheric pressure, and with dry equipment. The manner in which such coating of stent 14 may be carried out is illustrated in FIG. 4a. As shown in FIG. 4a, stent 14 may be immersed in a vessel 30 into the molten polymer 32 and withdrawn in order to coat the stent. The time in which stent 14 must remain immersed in liquid 32 varies depending on the construction of stent 14 and the chemical composition of liquid 32. However, in most cases, an immersion time of 10-15 seconds will be sufficient to obtain uniform deposition of the coating 20 on the wire members 18 of stent 14 (FIG. 3b). After stent 14 has been removed from liquid 32, it will be permitted to air dry such that a dry coating 20 remains deposited upon the outer surface of each wire 18 of stent 14.

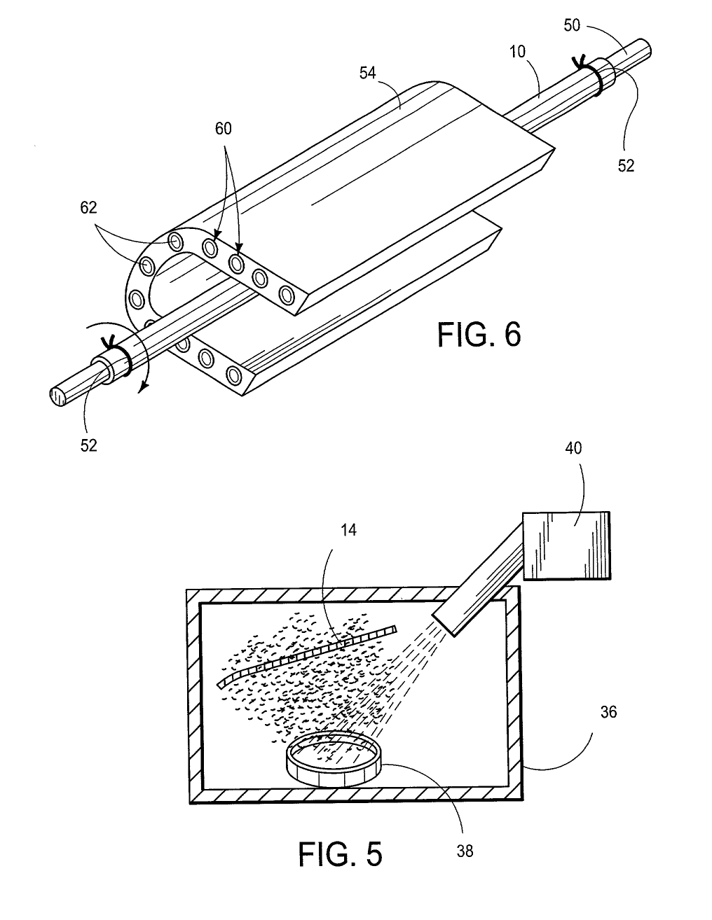

Optionally, after the air drying has been completed, coated stent 14 may be subjected to electron beam deposition, as illustrated in FIG. 5, to enhance the bonding of coating 20 to wire members 18 of stent 14. In accordance with this alternative deposition method, stent 14 is positioned within a closed vacuum chamber 36 wherein a mass comprising a composite of at least one polymer and at least one therapeutic substance 38 is located. An electron beam apparatus 40 is then utilized to project electron beam radiation onto mass 38 within the chamber 36 so as to cause sublimation of mass 38 and resultant deposition of layer 20 on the outer surface of stent 14. The apparatus and specific methodology useable to perform this electron beam deposition of coating 20 are well known to those of skill in the relevant art.

D. Assembly and Construction of the Integrally Drug Eluting Stented PTFE Graft

FIGS. 4b-4f show, in step-wise fashion, the preferred method for assembling and constructing graft 10.

As shown in FIG. 4b, tubular base graft 12 is initially disposed on a rod or mandrel 50. Mandrel 50 may comprise a stainless steel rod having an outer diameter that is only slightly smaller than the inner diameter of graft 12. In this manner, graft 12 may be slidably advanced onto the outer surface of mandrel 50 without undue effort or damage. Thereafter, coated stent 14 is axially advanced onto the outer surface of graft 12, as shown in FIG. 4c.

Thereafter, as shown in FIG. 4d, PTFE tape 17 is helically wrapped in a first direction in overlapping fashion on the outer surface of stent 14. In the preferred embodiment, tape of ½ inch width is used. The tape is helically wrapped about the stent at a pitch angle whereby 6 to 8 revolutions of the tape are applied per linear inch of stent 14. Thereafter, as shown in FIG. 4e, a second tape wrap in the opposite direction is accomplished, preferably using the same width of tape at the same pitch angle, thereby applying another 6-8 revolutions of tape 17 per linear inch of stent 14. In this manner, both wrappings of tape 17 (FIGS. 4d and 4e) combine to form a tubular, outer PTFE layer 16 which preferably has a thickness of less than 0.1 inches, and which may be formed of 1 to 10 consecutive (e.g., laminated) layers of the tape 17, for example, when using ePTFE tape of less than 1.6 g/cc density and ½ inch width, the first helical wrap (FIG. 4d) may deposit four consecutive layers of tape 17 and the second helical wrap (FIG. 4e) may deposit an additional 4 layers of tape 17, thereby resulting in an outer tubular layer 16 which is made up of a total of 8 layers of tape 17.

Optionally, to further promote bonding of the outer tubular layer 16 to stent 14 and/or inner base graft 12, liquid PTFE dispersion may be sprayed, painted or otherwise applied to and dried upon tape 17 prior to wrapping, or such liquid PTFE dispersion may be deposited by any suitable means (spraying, painting, etc.) between the outer tubular layer 16 formed by helically wrapped tape 17 and inner base graft 12. Or such liquid PTFE dispersion may be sprayed onto or otherwise applied to the outer surface of helically wrapped tape 17 such the small particles of PTFE contained within the liquid dispersion will migrate inwardly through pores in the layers of tape 17, and will thereby become deposited between outer tubular layer 16 and inner base graft 12 prior to subsequent heating of the assembly, as described below.

Thereafter, as shown in FIG. 4f, ligatures 52 of stainless steel wire are tied about the opposite ends of graft 10 so as to securely hold base graft 12, coated stent 14 and outer layer 16 on the mandrel 50. The mandrel having graft 10 disposed thereon is then heated using a “waffle-iron” heater, schematically shown in FIG. 4f, wherein heat is applied only to areas that correspond to the spaces not occupied by stent 14. The purpose of the “waffle-iron” heater is to avoid heating the drug covering the stent to its decomposition temperature. Heating causes outer PTFE layer 16 to heat fuse to inner base graft 12 through the openings 19 which exist in stent 14. In this manner, the desired integrally-drug eluting stented PTFE tubular graft 10 is formed. An alternative to the “waffle-iron” heater is to use a laser beam controlled by a computer to “hit” only the areas corresponding to the openings 19 which exist in stent 14. Computer controlled laser beams to accomplish such a purpose are known in the art.

E. Assembly and Construction of Internally Drug Eluting Stented Tube Graft

In one embodiment of the invention, inner base graft 12 is eliminated, thereby providing a drug eluting stented graft 10 comprising only stent 14 and outer tubular layer 16. This embodiment is of particular utility in connection with reducing the tendency of tissue ingrowth into the stent in certain applications. Thus, therapeutic agents including, sirolimus, paclitaxel, brachytherapeutic agents, and the like may be incorporated into the stent as taught by the invention to avoid such ingrowth. These stents are of particular importance as trans-hepatic stents, where such ingrowth is an important problem.

Here, the above-described manufacturing method is performed as described without tubular base graft 12, thereby forming a modified version of drug eluting stented graft 10 wherein outer tubular layer 16 is fused only to stent 14. In these embodiments stent 14 is coated with a lubricious polymer coating to provide lubricity and biocompatibility, which renders the graft suitable for use in applications wherein the exposed stent 14 will come in direct contact with biological fluid or blood. Thus, this embodiment of the present invention includes all possible arrangements wherein only outer tubular layer 16 is utilized in conjunction with stent 14, to provide an internally drug eluting stented graft 10 which is devoid of any internal tubular base graft 12.

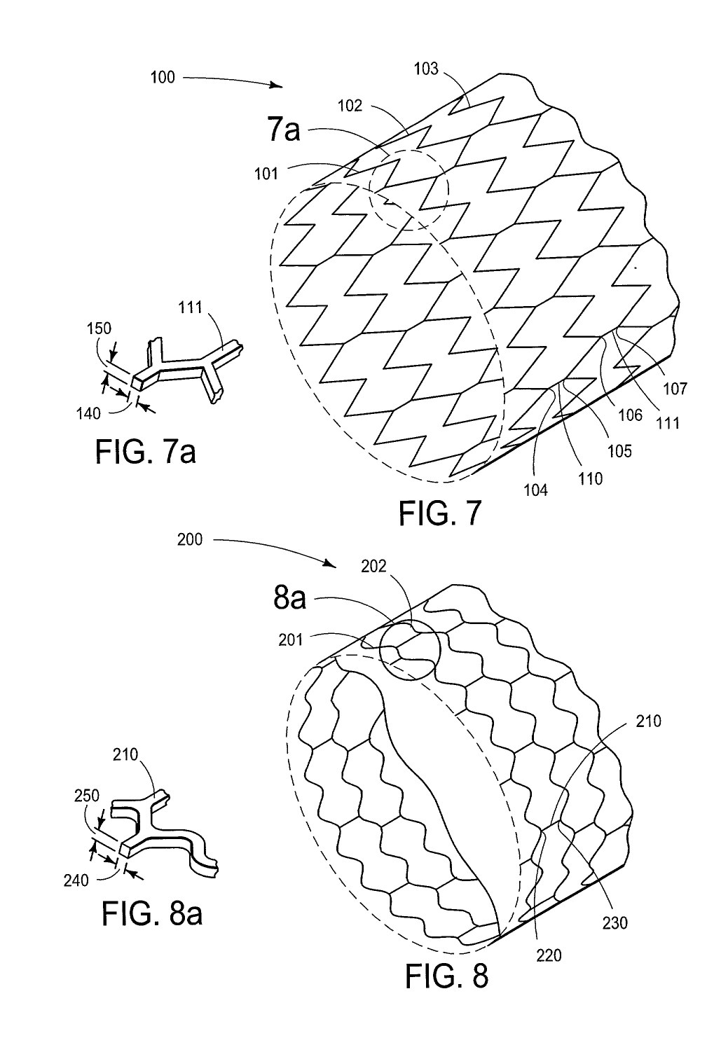

Referring now to FIG. 7 and FIG. 8, there are shown portions of two embodiments of the stent of the invention. They comprise a plurality of elements, wherein each element comprises an undulating shape formed into a generally cylindrical configuration having a cylinder axis, wherein each element is connected to an adjacent neighbor element by at least one linear connector. In FIG. 7, a portion of one embodiment of the stent is shown generally at 100. Stent portion 100 consists of three elements 101, 102 and 103, each of which comprises a zigzag pattern comprising a plurality of zigs having tips and a plurality of zags having tips. A tip 104 on a zig of element 101 and a nearest tip 105 of a zag of an adjacent neighbor element 105 generally lie in a plane passing through the cylinder axis, and are connected by a linear connector 105. Likewise, a tip 106 on a zig of element 102 and a nearest tip 107 on a zag of an adjacent neighbor element 103 generally lie in a plane passing through the cylinder axis, and are connected by a linear connector 111. Connector 111 is substantially circumferentially offset from adjacent neighbor connector 105. Stent 100 is constructed of material that has a width dimension 140 and a depth dimension 150 each of which is smaller than the length dimension of linear connectors 110 and 111. In FIG. 8, a portion of another embodiment of the stent is shown generally at 200. Stent portion 200 consists of two elements 201 and 202, each of which comprises an undulating pattern comprising a plurality of peaks and valleys. A valley 220 on element 201 and a nearest peak 230 of adjacent neighbor element 202 generally lie in a plane passing through the cylinder axis, and are connected by a linear connector 210. Stent 200 is constructed of material that has a width dimension 240 and a depth dimension 250 each of which is smaller than the length dimension of connector 210.

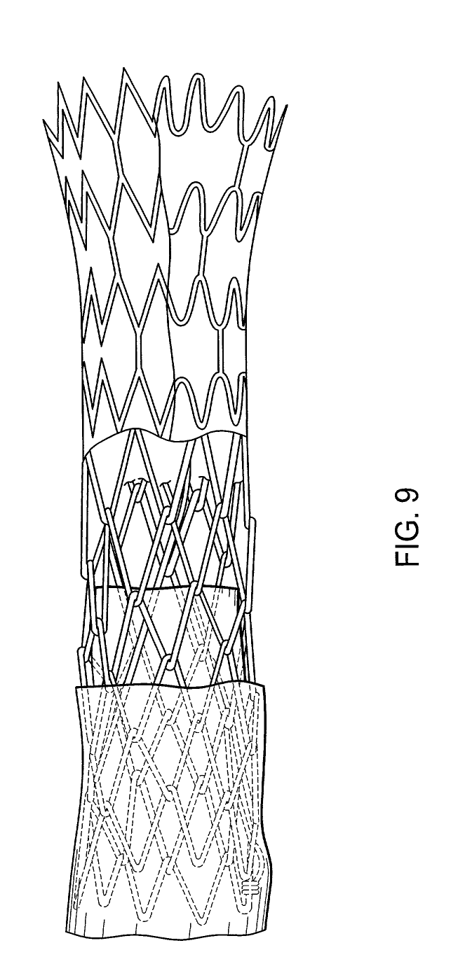

Uncoated stent designs comprising individual elements or wires and gaps or lateral openings are described in detail in U.S. Pat. Nos. 4,655,771 Wallsten); U.S. Pat. No. 4,954,126 (Wallsten); and U.S. Pat. No. 5,061,275 (Wallsten et al.), the entireties of which are hereby expressly incorporated herein by reference. An improved design combining these older features with the features shown in FIGS. 7 and 8, described above, is shown in the drug eluting radially expandable tubular stented graft shown generally at 290 in FIG. 9. Here, in the stent generally shown at 280, the wire and gap features of the older stent art, shown at 330 and 340 are combined elements having zigzag features, shown at 310, and sinusoidal features, shown at 300. All elements of the 310 and 300 type are connected using connectors as shown at 320. The resulting stent may be fabricated into any of the embodiments of the present invention.

In general, the invention comprises an improved stented graft that can alternately include a compact configuration having a first diameter and an expanded configuration having a greater diameter, comprising, in combination at least one stent formed in a generally cylindrical shape having an outer surface and a hollow bore extending longitudinally therethrough, wherein the stent can alternately exist in a compact configuration having a first diameter, and an expanded configuration having a greater diameter and a plurality of lateral openings; and, a flexible, porous, biocompatible tubular elastomer covering having a first end, a second end, an outer surface and a hollow bore that extends longitudinally therethrough to define an inner surface. The stent is deployed coaxially within the hollow bore of the covering such that the inner surface of the tubular covering is in contact with the outer surface of the stent.

Another embodiment is a tubular stented graft that is alternately deployable in a radially compact configuration having a first diameter and a radially expanded configuration having a second diameter. This stented graft includes a stent comprising at least one member formed in a generally cylindrical shape having an outer surface and a hollow bore which extends longitudinally therethrough to define an inner surface. The stent is initially radially collapsible to a diameter that is substantially equal to the first diameter of the stented graft, and subsequently radially expandable to a diameter which is substantially equal to the second diameter of the stented graft. A plurality of lateral openings exists in the stent when the stent is at its radially expanded second diameter. A continuous, tubular PTFE covering is formed on the stent, the PTFE covering comprising a tubular inner base graft formed of expanded, sintered PTFE. The tubular base graft has an outer surface and an inner surface, the tubular base graft being deployed coaxially within the hollow bore of the stent such that the outer surface of the tubular base graft is in contact with the inner surface of the stent, and the inner surface of the tubular base graft thereby defining a luminal passageway through the stented graft. A tubular outer layer is formed of expanded, sintered PTFE tape which has a width of less than about 1 inch, the tape having been wound about the outer surface of the stent to create the tubular outer layer thereon, such that the stent is captured between the outer layer and the tubular base graft. The tubular outer layer is attached to the tubular base graft through the lateral openings in the stent to form an integrally stented, continuous PTFE tube which is alternately disposable in the radially compact configuration of the first diameter and the radially expanded configuration of the second diameter.

The improvement comprises the device wherein the stent is coated with a coat comprising a composite of at least one biocompatible, pharmaceutically acceptable, bioerodible polymer and at least one therapeutic substance to form a drug eluting stented graft. The polymer may be a polyester. The therapeutic agent may be selected from the group consisting of antiplatelet agents, anticoagulant agents, antimetabolic agents, vasoactive agents, nitric oxide releasing agents, anti-inflammatory agents, antiproliferative agents, antisense agents, pro-endothelial agents, anti-migratory agents, antimicrobial agents, selective gene delivery vectors, sirolimus, actinomycin-D and paclitaxel. The selective gene delivery vectors may include Semliki Forest Virus (SMV) adapted to deliver restenosis preventing genes.

The polymer may be a hydrophobic, bioerodible, copolymer comprising mers I and II according to the following formula wherein:

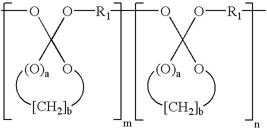

R₁ is a member selected from the group consisting of alkylene of 1 to 10 carbons; alkenylene of 2 to 10 carbons; alkyleneoxy of 2 to 6 carbons; cycloalkylene of 3 to 7 carbons; cycloalkylene of 3 to 7 carbons substituted with a member selected from the group consisting of alkyl of 1 to 7 carbons, an alkoxy of 1 to 7 carbons, an alkylene of 1 to 10 carbons, and an alkenyl of 2 to 7 carbons; cycloalkenylene of 4 to 7 carbons; cycloalkenylene of 4 to 7 carbons substituted with an alkyl of 1 to 7 carbons, an alkoxy of 1 to 7 carbons, an alkylene of 1 to 10 carbons, and an alkenyl of 2 to 7 carbons; arylene; and arylene substituted with an alkyl of 1 to 7 carbons, an alkoxy of 1 to 7 carbons, an alkylene of 1 to 10 carbons, an alkenyl of 2 to 7 carbons; and wherein a is 0 to 1; b is 2 to 6; m is greater than 10; n is greater than 10; and at least one of R₁, a, and b in mer I is different than R₁, a, and b in mer II; and wherein:

a composite of at least one polymer and at least one therapeutic substance when in operation bioerodes and releases the at least one therapeutic substance at a rate selected from (1) a zero order rate,(2) a continuous rate, and (3) a variable rate, which rate is produced by preselecting the composite of at least one polymer and at least one therapeutic substance, and the elastomer to give the desired result.

Alternatively, the at least one polymer may be a hydrophobic, bioerodible, terpolymer comprising mers I, II, and III according to the following formula, wherein:

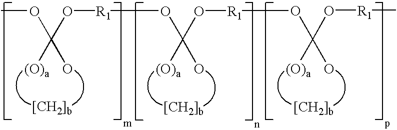

R₁ is a member selected from the group consisting of alkylene of 1 to 10 carbons; alkenylene of 2 to 10 carbons; alkyleneoxy of 2 to 6 carbons; cycloalkylene of 3 to 7 carbons; cycloalkylene of 3 to 7 carbons substituted with a member selected from the group consisting of alkyl of 1 to 7 carbons, alkoxy of 1 to 7 carbons, an alkylene of 1 to 10 carbons, and an alkenyl of 2 to 7 carbons; cycloalkenylene of 4 to 7 carbons; cycloalkenylene of 4 to 7 carbons substituted with an alkyl of 1 to 7 carbons, an alkoxy of 1 to 7 carbons, an alkylene of 1 to 10 carbons, and an alkenyl of 2 to 7 carbons; arylene; and arylene substituted with an alkyl of 1 to 7 carbons, an alkoxy of 1 to 7 carbons, an alkylene of 1 to 10 carbons, an alkenyl of 2 to 7 carbons; and wherein a is 0 to 1; b is 2 to 6; m is greater than 10; n is greater than 10; p is greater than 10; and at least one of R₁, a, and b in mers I, II and III is different than R₁, a, and b in mers I, II and III. The composite of at least one polymer and at least one therapeutic substance when in operation bioerodes and releases the at least one therapeutic substance at a rate selected from (1) a zero order rate,(2) a continuous rate, and (3) a variable rate, which rate is produced by preselecting the composite of the at least one polymer and the at least one therapeutic substance, and the elastomer to give the desired result. The drug eluting stented graft may include a multiplicity of microcapsules dispersed within the at least one polymer. The microcapsules have a wall formed of a drug release rate controlling material and therapeutic substance is contained within the multiplicity of microcapsules. The at least one polymer may have the formula:

[chemical structure]

wherein R₁ is a member selected from the group of divalent, trivalent and tetravalent radicals consisting of alkylene of 1 to 10 carbons; alkenylene of 2 to 10 carbons; alkyleneoxy of 2 to 6 carbons; cycloalkylene of 3 to 7 carbons; cycloalkylene of 3 to 7 carbons substituted with an alkyl of 1 to 7 carbons, alkoxy of 1 to 7 carbons, an alkylene of 1 to 10 carbons, and an alkenyl of 2 to 7 carbons; cycloalkenylene of 4 to 7 carbons cycloalkenylene of 4 to 7 carbons substituted with an alkyl of 1 to 7 carbons, an alkoxy of 1 to 7 carbons, an alkylene of 1 to 10 carbons, and an alkenyl of 2 to 7 carbons; arylene; and arylene substituted with an alkyl of 1 to 7 carbons, an alkoxy of 1 to 7 carbons, and an alkenyl of 2 to 7 carbons; R₂ and R₃ are selected from the group consisting of alkyl of 1 to 7 carbons; alkenyl of 2 to 7 carbons; alkoxy of 1 to 7 carbons; alkenyloxy of 2 to 7 carbons; alkylene of 2 to 6 carbons; alkenylene of 3 to 6 carbons; alkyleneoxy of 2 to 6 carbons; alkenyleneoxy of 3 to 6 carbons; aryloxy; aralkyleneoxy of 8 to 12 carbons; aralkenyleneoxy of 8 to 12 carbons; oxa; OR₁O with R₁ as defined above; a heterocyclic ring of 5 to 8 carbon and oxygen atoms formed when R₂ and R₃ are taken together; a heterocyclic ring of 5 to 8 carbon and oxygen atoms substituted with an alkyl of 1 to 7 carbons, an alkoxy of 1 to 7 carbons and alkenyl of 2 to 7 carbons formed when R₂ and R₃ are taken together; a fused polycyclic ring of 8 to 12 carbon and oxygen atoms formed when R₂ and R₃ are taken together; a fused polycyclic ring of 8 to 12 carbon and oxygen atoms substituted with an alkyl of 1 to 7 carbons; an alkoxy of 1 to 7 carbons and an alkenyl of 2 to 7 carbons; and wherein at least one of the R₂ and R₃ is a member selected from the group consisting of alkoxy, alkenyloxy and OR₁O; R₂ and R₃ when taken together are a member selected from the group of heterocyclic and fused polycyclic rings having at least one oxygen atom in the ring; and wherein n is greater than 10.

In operation, the polymer and the microcapsules bioerode at a controlled and continuous rate over a prolonged period of time, thereby releasing the at least one therapeutic substance at a controlled and continuous rate over a prolonged period of time.

The coat of the stent of the drug eluting stented graft may further comprise at least a first layer and a second layer, wherein the first layer comprises the at least one therapeutic substance and at least a first polymer, and the second layer comprises the at least one therapeutic substance and at least a second polymer. At least one of the first polymer and the second polymer are selected from the group consisting of polymers of the formula:

[chemical structure]

wherein R₁ is a member selected from the group of divalent, trivalent and tetravalent radicals consisting of alkylene of 1 to 10 carbons; alkenylene of 2 to 10 carbons; alkyleneoxy of 2 to 6 carbons; cycloalkylene of 3 to 7 carbons; cycloalkylene of 3 to 7 carbons substituted with an alkyl of 1 to 7 carbons, alkoxy of 1 to 7 carbons, alkylene of 1 to 10 carbons, and an alkenyl of 2 to 7 carbons; cycloalkenylene of 4 to 7 carbons; cycloalkenylene of 4 to 7 carbons substituted with an alkyl of 1 to 7 carbons, an alkoxy of 1 to 7 carbons, an alkylene of 1 to 10 carbons, and an alkenyl of 2 to 7 carbons; arylene; and arylene substituted with an alkyl of 1 to 7 carbons, an alkoxy of 1 to 7 carbons, and an alkenyl of 2 to 7 carbons; R₂ and R₃ are selected from the group consisting of alkyl of 1 to 7 carbons; alkenyl of 2 to 7 carbons; alkoxy of 1 to 7 carbons; alkenyloxy of 2 to 7 carbons; alkylene of 2 to 6 carbons; alkenylene of 3 to 6 carbons; alkyleneoxy of 2 to 6 carbons; alkenyleneoxy of 3 to 6 carbons; aryloxy; aralkyleneoxy of 8 to 12 carbons; aralkenyleneoxy of 8 to 12 carbons; oxa; OR₁O with R₁ as defined above; a heterocyclic ring of 5 to 8 carbon and oxygen atoms formed when R₂ and R₃ are taken together; a heterocyclic ring of 5 to 8 carbon and oxygen atoms substituted with an alkyl of 1 to 7 carbons; an alkoxy of 1 to 7 carbons and an alkenyl of 2 to 7 carbons formed when R₂ and R₃ are taken together; a fused polycyclic ring of 8 to 12 carbon and oxygen atoms formed when R₂ and R₃ are taken together; a fused polycyclic ring of 8 to 12 carbon and oxygen atoms substituted with an alkyl of 1 to 7 carbons, an alkoxy of 1 to 7 carbons and an alkenyl of 2 to 7 carbons; and wherein at least one of the R₂ and R₃ is a member selected from the group consisting of alkoxy, alkenyloxy and OR₁O; R₁ and R₃when taken together are a member selected from the group of heterocyclic and fused polycyclic rings having at least one oxygen atom in the ring; and wherein is greater than 10 In operation, the layers bioerode at a controlled and continuous rate over a prolonged period of time, thereby releasing the at least one therapeutic substance at a controlled and continuous rate over a prolonged period of time. In this case, the first polymer may be a pharmaceutically acceptable biocompatible non-bioerodible polymer that sequesters an agent, such as palladium-103 (¹⁰³Pd), ¹⁹²Ir, ³²P, ¹⁸⁸Re, and Sr/Y90 source trains, for brachytherapy.

The drug eluting stented graft may have a multiplicity of discrete, closed cells within the at least one polymer, the cells having a wall formed and defined by the at least one polymer. The polymer has the formula:

[chemical structure]

wherein R₁ is a member selected from the group of divalent, trivalent and tetravalent radicals consisting of alkylene of 1 to 10 carbons; alkenylene of 2 to 10 carbons; alkyleneoxy of 2 to 6 carbons; cycloalkylene of 3 to 7 carbons; cycloalkylene of 3 to 7 carbons substituted with an alkyl of 1 to 7 carbons, alkoxy of 1 to 7 carbons, an alkylene of 1 to 10 carbons, and an alkenyl of 2 to 7 carbons; cycloalkenylene of 4 to 7 carbons; cycloalkenylene of 4 to 7 carbons substituted with an alkyl of 1 to 7 carbons, an alkoxy of 1 to 7 carbons, an alkylene of 1 to 10 carbons, and an alkenyl of 2 to 7 carbons; arylene; and arylene substituted with an alkyl of 1 to 7 carbons, an alkoxy of 1 to 7 carbons, and an alkenyl of 2 to 7 carbons; R₂ and R₃ are selected from the group consisting of alkyl of 1 to 7 carbons; alkenyl of 2 to 7 carbons; alkoxy of 1 to 7 carbons; alkenyloxy of 2 to 7 carbons; alkylene of 2 to 6 carbons; alkenylene of 3 to 6 carbons; alkyleneoxy of 2 to 6 carbons; alkenyleneoxy of 3 to 6 carbons; aryloxy; aralkyleneoxy of 8 to 12 carbons; aralkenyleneoxy of 8 to 12 carbons; oxa; OR₁O with R₁ as defined above; a heterocyclic ring of 5 to 8 carbon and oxygen atoms formed when R₂ and R₃ are taken together; a heterocyclic ring of 5 to 8 carbon and oxygen atoms substituted with an alkyl of 1 to 7 carbons, an alkoxy of 1 to 7 carbons and an alkenyl of 2 to 7 carbons formed when R₂ and R₃ are taken together; a fused polycyclic ring of 8 to 12 carbon and oxygen atoms formed when R₂ and R₃ are taken together; a fused polycyclic ring of 8 to 12 carbon and oxygen atoms substituted with an alkyl of 1 to 7 carbons, an alkoxy of 1 to 7 carbons and an alkenyl of 2 to 7 carbons; and wherein at least one of the R₂ and R₃ is a member selected from the group consisting of alkoxy, alkenyloxy and OR₁O; R₂ and R₃ when taken together are a member selected from the group of heterocyclic and fused polycyclic rings having at least one oxygen atom in the ring; and wherein n is greater than 10.

The at least one therapeutic substance is dissolved in a pharmaceutically acceptable carrier that is a solvent for the at least one therapeutic substance and a nonsolvent for the at least one polymer is contained within the multiplicity of discrete, closed cells. When in operation, the at least one polymer is capable of bioeroding at a controlled and continuous rate over a prolonged period of time, thereby releasing the at least one therapeutic substance at a controlled and continuous rate over a prolonged period of time.

The stent comprises a plurality of elements. Each element comprises an undulating linear shape formed into a generally cylindrical configuration having a cylinder axis generally aligned on the axis of the hollow bore, and each element is connected to an adjacent neighbor element by at least one linear connector. The elements may comprise a spiral. One connector may be substantially circumferentially offset from an adjacent neighbor connector, and may form a helical array. Alternatively, a connector may not be substantially circumferentially offset from an adjacent neighbor connector.

The undulating linear shape may be a generally zigzag shape comprising a plurality of zigs having tips and a plurality of zags having tips, wherein the tip of each zig of each element and the nearest the tip of each zig of an adjacent neighbor element generally lie in a plane passing through the axis of the hollow bore, and wherein the tip of at least one zig of each element and at least one nearest tip of a zig of an adjacent neighbor are connected by one linear connector.

Alternatively, the undulating linear shape may be a sinusoidal shape having a plurality of peaks and a plurality of valleys. Each peak of each element and each valley of an adjacent neighbor may lie generally in a common plane passing through the axis of the hollow bore, and at least one peak of each element and the valley of an adjacent neighbor lying generally in the common plane may be connected by one linear connector. The length of each linear connector is greater than its width or depth, and may be 3-10 times greater than the width or depth.

The stent and elastomer may be anchored to each other by means for anchoring, such as protrusions of the covering that fixedly protrude into the lateral openings in the stent. The elastomer may be polytetrafluoroethylene, fluorinated ethylene propylene, polytetrafluoroethylene-perfluoroalkyl vinyl ether copolymer, polyvinyl chloride, polypropylene, polyethylene terephthalate, broad fluoride, other biocompatible plastics, and expanded, sintered PTFE (which may be tape) having fibrils measuring about 300μ-5μ in length. The tape may have a width of less than about 0.5 inches to about 1 inch, a thickness of less than 0.015 inch (0.038 cm.), and a density of less than 1.6 g/cc. The tape may be wound about the stent in overlapping fashion, for example, helically. The tape may be wound in a first direction and then in the opposite direction, and comprise 1 to 10 layers. The tape may be helically wrapped such that 6-8 revolutions of tape are applied per longitudinal inch (2.54 cm.) of the drug eluting stented graft. The thickness of the covering may be less than 0.1 inch (0.25 cm.)

The drug eluting stented graft may include a self-expanding stent comprising a shape memory alloy that can alternately exist in a first and a second crystalline state, or it may include a pressure-expandable stent. The stent may be formed of a metal alloy comprising at least two elements selected from the group consisting of iron, cobalt, chromium, nickel, titanium, niobium, and molybdenum. For example, the alloy may comprise at least about 51% to about 59% nickel and the remainder comprising titanium. Alternatively, it may comprise about 0.25% chromium, at least about 51% to about 59% nickel, and the remainder comprising titanium.

The composite coating of the drug eluting stented graft may be applied to the stent by the steps of immersing the stent in a liquid dispersion of the composite, removing the stent from the liquid dispersion of the composite, and drying the liquid dispersion of the composite that has remained on the stent. The composite coating may be formed by electron beam deposition, and the tubular covering may be adherent to the coat.

A method for the treatment of cardiovascular disease, comprises implanting the drug eluting stented graft in a patient in need of such treatment wherein the implantation is effective to ameliorate one or more of the symptoms of the cardiovascular disease. An article of manufacture, comprises packaging material and the drug eluting stented graft contained within the packaging material, wherein the drug eluting stented graft is effective for implantation in a patient afflicted with cardiovascular disease, and the packaging material includes a label that indicates that the device is effective for said implantation.

It will be appreciated that the invention has been described with reference to certain presently preferred embodiments of the invention. Various additions, deletions, alterations and modifications may be made to the above-described embodiments without departing from the intended spirit and scope of the invention. For example, the linear connectors may collectively form arrays that may be helical, linear, or neither helical nor linear. Likewise, linear connectors may connect peaks to peaks, valleys to valleys, or peaks to valleys. Again, linear connectors may connect zigs to zigs, zags to zags, or zigs to zags. Accordingly, it is intended that all such reasonable additions, deletions, modifications and alterations to the above described embodiments be included within the scope of the following claims.

On this basis, the instant invention should be recognized as constituting progress in science and the useful arts, as solving the problems in cardiology enumerated above. In the foregoing description, certain terms have been used for brevity, clearness and understanding, but no unnecessary limitation are to be implied therefrom beyond the requirements of the prior art, because such words are used for descriptive purposes herein and are intended to be broadly construed.

Having described preferred embodiments of the invention with reference to the accompanying drawings, it is to be understood that the invention is not limited to those precise embodiments, and that the various changes and modifications may be effected therein by one skilled in the art without departing from the scope or spirit of the invention s defined in the appended claims. For example, the product can have other shapes, or could make use of other metals and plastics. Thus, the scope of the invention should be determined by the appended claims and their legal equivalents, rather than by the examples given. All changes that come within the meaning and range of equivalency of the claims are to be embraced within their scope.

### Definitions

Unless defined otherwise, all technical and scientific terms used herein have the same meaning as is commonly understood by one of skill in the art to which this invention belongs. All patents and publications referred to herein are incorporated in their entirety by reference.

What is claimed is:

1. In a stented graft that can alternately include a compact configuration having a first diameter and an expanded configuration having a greater diameter, comprising, in combination:
at least one stent formed in a generally cylindrical shape having an outer surface and a hollow bore extending longitudinally therethrough, wherein said stent can alternately exist in a compact configuration having a first diameter, and an expanded configuration having a greater diameter and a plurality of lateral openings; and,
a flexible, porous, biocompatible tubular elastomer covering having a first end, a second end, an outer surface and a hollow bore that extends longitudinally therethrough to define an inner surface;
said stent being deployed coaxially within said hollow bore of said covering such that said inner surface of said tubular covering is in contact with said outer surface of said stent;
the improvement wherein said stent is coated with a coat comprising a composite of at least one polymer and at least one therapeutic substance to form a drug eluting stented graft.

2. The drug eluting stented graft of claim 1, wherein said at least one polymer is a biocompatible, pharmaceutically acceptable, bioerodible polymer.

3. The drug eluting stented graft of claim 1, wherein said at least one polymer is a polyester.

4. The drug eluting stented graft of claim 1, wherein said at least one therapeutic agent is selected from the group consisting of antiplatelet agents, anticoagulant agents, antimetabolic agents, vasoactive agents, nitric oxide releasing agents, anti-inflammatory agents, antiproliferative agents, antisense agents, pro-endothelial agents, anti-migratory agents, antimicrobial agents, selective gene delivery vectors, sirolimus, actinomycin-D and paclitaxel.

5. The drug eluting stented graft of claim 4, wherein said selective gene delivery vectors are Semliki Forest Virus (SMV) adapted to deliver restenosis preventing genes.

6. The drug eluting stented graft of claim 1, wherein said at least one polymer is a hydrophobic, bioerodible, copolymer comprising mers I and II according to the following formula: 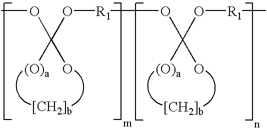
wherein:
R₁ is a member selected from the group consisting of alkylene of 1 to 10 carbons; alkenylene of 2 to 10 carbons; alkyleneoxy of 2 to 6 carbons; cycloalkylene of 3 to 7 carbons; cycloalkylene of 3 to 7 carbons substituted with a member selected from the group consisting of alkyl of 1 to 7 carbons, an alkoxy of 1 to 7 carbons, an alkylene of 1 to 10 carbons, and an alkenyl of 2 to 7 carbons; cycloalkenylene of 4 to 7 carbons; cycloalkenylene of 4 to 7 carbons substituted with an alkyl of 1 to 7 carbons, an alkoxy of 1 to 7 carbons, an alkylene of 1 to 10 carbons, and an alkenyl of 2 to 7 carbons; arylene; and arylene substituted with an alkyl of 1 to 7 carbons, an alkoxy of 1 to 7 carbons, an alkylene of 1 to 10 carbons, an alkenyl of 2 to 7 carbons; and wherein a is 0 to 1; b is 2 to 6; m is greater than 10; n is greater than 10; and at least one of R₁, a, and b in mer I is different than R₁, a, and b in mer II; and wherein:
said composite of at least one polymer and at least one therapeutic substance when in operation bioerodes and releases said at least one therapeutic substance at a rate selected from (1) a zero order rate,(2) a continuous rate, and (3) a variable rate, which rate is produced by preselecting said composite of at least one polymer and at least one therapeutic substance, and said elastomer to give the desired result.

7. The drug eluting stented graft of claim 1, wherein said at least one polymer is a hydrophobic, bioerodible, terpolymer comprising mers I, II, and III according to the following formula: 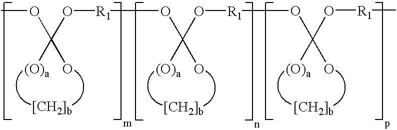
wherein:
R₁ is a member selected from the group consisting of alkylene of 1 to 10 carbons; alkenylene of 2 to 10 carbons; alkyleneoxy of 2 to 6 carbons; cycloalkylene of 3 to 7 carbons; cycloalkylene of 3 to 7 carbons substituted with a member selected from the group consisting of alkyl of 1 to 7 carbons, alkoxy of 1 to 7 carbons, an alkylene of 1 to 10 carbons, and an alkenyl of 2 to 7 carbons; cycloalkenylene of 4 to 7 carbons; cycloalkenylene of 4 to 7 carbons substituted with an alkyl of 1 to 7 carbons, an alkoxy of 1 to 7 carbons, an alkylene of 1 to 10 carbons, and an alkenyl of 2 to 7 carbons; arylene; and arylene substituted with an alkyl of 1 to 7 carbons, an alkoxy of 1 to 7 carbons, an alkylene of 1 to 10 carbons, an alkenyl of 2 to 7 carbons; and wherein a is 0 to 1; b is 2 to 6; m is greater than 10; n is greater than 10; p is greater than 10; and at least one of R₁, a, and b in mers I, II and III is different than R₁, a, and b in mers I, II and III; and wherein:
said composite of at least one polymer and at least one therapeutic substance when in operation bioerodes and releases said at least one therapeutic substance at a rate selected from (1) a zero order rate,(2) a continuous rate, and (3) a variable rate, which rate is produced by preselecting said composite of said at least one polymer and said at least one therapeutic substance, and said elastomer to give the desired result.

8. The drug eluting stented graft of claim 1, wherein:
a multiplicity of microcapsules is dispersed within said at least one polymer, wherein said microcapsules have a wall formed of a drug release rate controlling material;
said at least one therapeutic substance is contained within said multiplicity of microcapsules; and,
said at least one polymer has the formula: [chemical structure]
wherein R₁ is a member selected from the group of divalent, trivalent and tetravalent radicals consisting of alkylene of 1 to 10 carbons; alkenylene of 2 to 10 carbons; alkyleneoxy of 2 to 6 carbons; cycloalkylene of 3 to 7 carbons; cycloalkylene of 3 to 7 carbons substituted with an alkyl of 1 to 7 carbons, alkoxy of 1 to 7 carbons, an alkylene of 1 to 10 carbons, and an alkenyl of 2 to 7 carbons; cycloalkenylene of 4 to 7 carbons cycloalkenylene of 4 to 7 carbons substituted with an alkyl of 1 to 7 carbons, an alkoxy of 1 to 7 carbons, an alkylene of 1 to 10 carbons, and an alkenyl of 2 to 7 carbons; arylene; and arylene substituted with an alkyl of 1 to 7 carbons, an alkoxy of 1 to 7 carbons, and an alkenyl of 2 to 7 carbons; R₂ and R₃ are selected from the group consisting of alkyl of 1 to 7 carbons; alkenyl of 2 to 7 carbons; alkoxy of 1 to 7 carbons; alkenyloxy of 2 to 7 carbons; alkylene of 2 to 6 carbons; alkenylene of 3 to 6 carbons; alkyleneoxy of 2 to 6 carbons; alkenyleneoxy of 3 to 6 carbons; aryloxy; aralkyleneoxy of 8 to 12 carbons; aralkenyleneoxy of 8 to 12 carbons; oxa; OR₁O with R₁ as defined above; a heterocyclic ring of 5 to 8 carbon and oxygen atoms formed when R₂ and R₃ are taken together; a heterocyclic ring of 5 to 8 carbon and oxygen atoms substituted with an alkyl of 1 to 7 carbons, an alkoxy of 1 to 7 carbons and alkenyl of 2 to 7 carbons formed when R₂ and R₃ are taken together; a fused polycyclic ring of 8 to 12 carbon and oxygen atoms formed when R₂ and R₃ are taken together; a fused polycyclic ring of 8 to 12 carbon and oxygen atoms substituted with an alkyl of 1 to 7 carbons; an alkoxy of 1 to 7 carbons and an alkenyl of 2 to 7 carbons; and wherein at least one of said R₂ and R₃ is a member selected from the group consisting of alkoxy, alkenyloxy and OR₁O; R₂ and R₃ when taken together are a member selected from the group of heterocyclic and fused polycyclic rings having at least one oxygen atom in the ring; and wherein n is greater than 10;
so that, in operation, said polymer and said microcapsules bioerode at a controlled and continuous rate over a prolonged period of time, thereby releasing said at least one therapeutic substance at a controlled and continuous rate over a prolonged period of time.

9. The drug eluting stented graft of claim 1, wherein:
said coat further comprises at least a first layer and a second layer, wherein said first layer comprises said at least one therapeutic substance and at least a first polymer, and said second layer comprises said at least one therapeutic substance and at least a second polymer, wherein at least one of said first polymer and said second polymer are selected from the group consisting of polymers of the formula: [chemical structure]
wherein R₁ is a member selected from the group of divalent, trivalent and tetravalent radicals consisting of alkylene of 1 to 10 carbons; alkenylene of 2 to 10 carbons; alkyleneoxy of 2 to 6 carbons; cycloalkylene of 3 to 7 carbons; cycloalkylene of 3 to 7 carbons substituted with an alkyl of 1 to 7 carbons, alkoxy of 1 to 7 carbons, alkylene of 1 to 10 carbons, and an alkenyl of 2 to 7 carbons; cycloalkenylene of 4 to 7 carbons; cycloalkenylene of 4 to 7 carbons substituted with an alkyl of 1 to 7 carbons, an alkoxy of 1 to 7 carbons, an alkylene of 1 to 10 carbons, and an alkenyl of 2 to 7 carbons; arylene; and arylene substituted with an alkyl of 1 to 7 carbons, an alkoxy of 1 to 7 carbons, and an alkenyl of 2 to 7 carbons; R₂ and R₃ are selected from the group consisting of alkyl of 1 to 7 carbons; alkenyl of 2 to 7 carbons; alkoxy of 1 to 7 carbons; alkenyloxy of 2 to 7 carbons; alkylene of 2 to 6 carbons; alkenylene of 3 to 6 carbons; alkyleneoxy of 2 to 6 carbons; alkenyleneoxy of 3 to 6 carbons; aryloxy; aralkyleneoxy of 8 to 12 carbons; aralkenyleneoxy of 8 to 12 carbons; oxa; OR₁O with R₁ as defined above; a heterocyclic ring of 5 to 8 carbon and oxygen atoms formed when R₂ and R₃ are taken together; a heterocyclic ring of 5 to 8 carbon and oxygen atoms substituted with an alkyl of 1 to 7 carbons; an alkoxy of 1 to 7 carbons and an alkenyl of 2 to 7 carbons formed when R₂ and R₃ are taken together; a fused polycyclic ring of 8 to 12 carbon and oxygen atoms formed when R₂ and R₃ are taken together; a fused polycyclic ring of 8 to 12 carbon and oxygen atoms substituted with an alkyl of 1 to 7 carbons, an alkoxy of 1 to 7 carbons and an alkenyl of 2 to 7 carbons; and wherein at least one of said R₂ and R₃ is a member selected from the group consisting of alkoxy, alkenyloxy and OR₁O; R₁ and R₃ when taken together are a member selected from the group of heterocyclic and fused polycyclic rings having at least one oxygen atom in the ring; and wherein is greater than 10;
so that when in operation, said layers bioerode at a controlled and continuous rate over a prolonged period of time, thereby releasing said at least one therapeutic substance at a controlled and continuous rate over a prolonged period of time.

10. The drug eluting stented graft of claim 9, wherein said first polymer is a pharmaceutically acceptable biocompatible non-bioerodible polymer that sequesters an agent for brachytherapy.

11. The drug eluting stented graft of claim 10, wherein said agent for brachytherapy is selected from the group consisting of palladium-103 (¹⁰³Pd), ¹⁹²Ir, ³²P, ¹⁸⁸Re, and Sr/Y90 source trains.

12. The drug eluting stented graft of claim 1, wherein:
a multiplicity of discrete, closed cells exists within said at least one polymer, said cells having a wall formed and defined by said at least one polymer;
said at least one polymer has the formula: 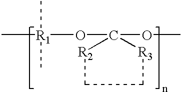
wherein R₁ is a member selected from the group of divalent, trivalent and tetravalent radicals consisting of alkylene of 1 to 10 carbons; alkenylene of 2 to 10 carbons; alkyleneoxy of 2 to 6 carbons; cycloalkylene of 3 to 7 carbons; cycloalkylene of 3 to 7 carbons substituted with an alkyl of 1 to 7 carbons, alkoxy of 1 to 7 carbons, an alkylene of 1 to 10 carbons, and an alkenyl of 2 to 7 carbons; cycloalkenylene of 4 to 7 carbons; cycloalkenylene of 4 to 7 carbons substituted with an alkyl of 1 to 7 carbons, an alkoxy of 1 to 7 carbons, an alkylene of 1 to 10 carbons, and an alkenyl of 2 to 7 carbons; arylene; and arylene substituted with an alkyl of 1 to 7 carbons, an alkoxy of 1 to 7 carbons, and an alkenyl of 2 to 7 carbons; R₂ and R₃ are selected from the group consisting of alkyl of 1 to 7 carbons; alkenyl of 2 to 7 carbons; alkoxy of 1 to 7 carbons; alkenyloxy of 2 to 7 carbons; alkylene of 2 to 6 carbons; alkenylene of 3 to 6 carbons; alkyleneoxy of 2 to 6 carbons; alkenyleneoxy of 3 to 6 carbons; aryloxy; aralkyleneoxy of 8 to 12 carbons; aralkenyleneoxy of 8 to 12 carbons; oxa; OR₁O with R₁ as defined above; a heterocyclic ring of 5 to 8 carbon and oxygen atoms formed when R₂ and R₃ are taken together; a heterocyclic ring of 5 to 8 carbon and oxygen atoms substituted with an alkyl of 1 to 7 carbons, an alkoxy of 1 to 7 carbons and an alkenyl of 2 to 7 carbons formed when R₂ and R₃ are taken together; a fused polycyclic ring of 8 to 12 carbon and oxygen atoms formed when R₂ and R₃ are taken together; a fused polycyclic ring of 8 to 12 carbon and oxygen atoms substituted with an alkyl of 1 to 7 carbons, an alkoxy of 1 to 7 carbons and an alkenyl of 2 to 7 carbons; and wherein at least one of said R₂ and R₃ is a member selected from the group consisting of alkoxy, alkenyloxy and OR₁O; R₂ and R₃when taken together are a member selected from the group of heterocyclic and fused polycyclic rings having at least one oxygen atom in the ring; and wherein n is greater than 10;
wherein said at least one therapeutic substance dissolved in a pharmaceutically acceptable carrier that is a solvent for said at least one therapeutic substance and a nonsolvent for said at least one polymer is contained within said multiplicity of discrete, closed cells;
so that, when in operation, said at least one polymer is capable of bioeroding at a controlled and continuous rate over a prolonged period of time, thereby releasing said at least one therapeutic substance at a controlled and continuous rate over a prolonged period of time.

13. The drug eluting stented graft of claim 1, wherein said stent comprises a plurality of elements, wherein each said element comprises an undulating linear shape formed into a generally cylindrical configuration having a cylinder axis generally aligned on the axis of said hollow bore, and wherein each said element is connected to an adjacent neighbor element by at least one linear connector.

14. The drug eluting stented graft of claim 1, wherein said plurality of elements comprises a spiral.

15. The drug eluting stented graft of claim 1, wherein at least one said connector is substantially circumferentially offset from an adjacent neighbor connector.

16. The drug eluting stented graft of claim 15, wherein said circumferentially offset connectors form a helical array.

17. The drug eluting stented graft of claim 1, wherein at least one said connector is not substantially circumferentially offset from an adjacent neighbor connector.

18. The drug eluting stented graft of claim 1, wherein said undulating linear shape is a generally zigzag shape comprising a plurality of zigs having tips and a plurality of zags having tips, wherein said tip of each said zig of each element and the nearest said tip of each said zig of an adjacent neighbor element generally lie in a plane passing through the axis of said hollow bore, and wherein said tip of at least one said zig of each element and at least one said nearest said tip of a zig of an adjacent neighbor are connected by one said linear connector.

19. The drug eluting stented graft of claim 1, wherein said undulating linear shape is a sinusoidal shape having a plurality of peaks and a plurality of valleys, wherein each said peak of each element and each said valley of an adjacent neighbor lie generally in a common plane passing through the axis of said hollow bore, and wherein at least one said peak of each element and said valley of an adjacent neighbor lying generally in said common plane are connected by one said linear connector.

20. The drug eluting stented graft of claim 1, wherein each said linear connector has a length dimension generally parallel to the axis of said hollow bore, and a width and depth dimension, and wherein said length dimension is greater than said width dimension and said length dimension is greater than said depth dimension.

21. The drug eluting stented graft of claim 20, wherein said length dimension is about 3 to 10 times greater than said width dimension, and said length dimension is about 3 to 10 times greater than said depth dimension.

22. The drug eluting stented graft according to claim 1, wherein said stent and said elastomer are anchored to each other by means for anchoring.

23. The tubular drug eluting stented graft according to claim 22, wherein said means for anchoring comprise protrusions of said covering that fixedly protrude into said lateral openings in said stent.

24. The drug eluting stented graft of claim 1 wherein said elastomer covering is formed of an elastomer selected from the group consisting of polytetrafluoroethylene, fluorinated ethylene propylene, polytetrafluoroethylene-perfluoroalkyl vinyl ether copolymer, polyvinyl chloride, polypropylene, polyethylene terephthalate, broad fluoride; and, other biocompatible plastics.

25. The drug eluting stented graft of claim 1 wherein said elastomer covering is formed of expanded, sintered PTFE tape, said tape having been wound about the outer surface of said stent to create said covering thereon.

26. The drug eluting stented graft of claim 24, wherein said polytetrafluoroethylene is expanded polytetrafluoroethylene having fibrils.

27. The drug eluting stented graft of claim 26, wherein said fibrils measure up to about 300μ in length.

28. The drug eluting stented graft of claim 26, wherein said fibrils measure up to about 200μ in length.

29. The drug eluting stented graft of claim 26, wherein said fibrils measure up to about 100μ in length.

30. The drug eluting stented graft of claim 26, wherein said fibrils measure up to about 50μ in length.

31. The drug eluting stented graft of claim 26, wherein said fibrils measure up to about 5μ in length.

32. The drug eluting stented graft of claim 25 wherein said tape has a width of less than about 1 inch.

33. The drug eluting stented graft of claim 25 wherein said tape has a thickness of less than 0.015 inch (0.038 cm.) and wherein said tape is wound about said stent in overlapping fashion, such that said elastomer covering comprises 1 to 10 layers of said tape.

34. The drug eluting stented graft of claim 25 wherein said tape is helically wrapped about said stent.

35. The drug eluting stented graft of claim 25 wherein said tape has a width of 0.5 inches (1.27 cm), and wherein said tape is helically wrapped such that 6-8 revolutions of tape are applied per longitudinal inch (2.54 cm.) of said drug eluting stented graft.

36. The drug eluting stented graft of claim 25 wherein said tape is helically wrapped alternately in a first direction and then in the opposite direction.

37. The drug eluting stented graft of claim 36 further comprising 8 layers of said tape.

38. The drug eluting stented graft of claim 1 wherein said stent is a self-expanding stent.

39. The drug eluting stented graft of claim 38, wherein said self-expanding stent comprises a shape memory alloy that can alternately exist in a first and a second crystalline state, wherein said stent assumes a radially expanded configuration when said shape memory alloy is in said first crystalline state, and a radially compact configuration when said shape memory alloy is in said second crystalline state.

40. The drug eluting stented graft of claim 1 wherein said stent is a pressure-expandable stent.

41. The drug eluting stented graft of claim 1 wherein said stent is formed of a metal alloy comprising at least two elements selected from the group consisting of iron, cobalt, chromium, nickel, titanium, niobium, and molybdenum.

42. The drug eluting stented graft of claim 39 wherein said shape memory alloy comprises at least about 51% to about 59% nickel and the remainder comprising titanium.

43. The drug eluting stented graft of claim 39 wherein said shape memory alloy comprises about 0.25% chromium, at least about 51% to about 59% nickel, and the remainder comprising titanium.

44. The drug eluting stented graft of claim 1 wherein said covering has a thickness of less than 0.1 inch (0.25 cm.).

45. The drug eluting stented graft of claim 25 wherein said PTFE tape has a thickness of less than 0.015 inches (0.038 cm.), said tape being wrapped about said stent in overlapping fashion so as to form said covering.

46. The drug eluting stented graft of claim 25 wherein said PTFE tape has a density of less than 1.6 g/cc.

47. The drug eluting stented graft of claim 25 wherein said covering has a thickness of less than 0.1 inch (0.25 cm.) and said PTFE tape has a density of less than 1.6 g/cc.

48. The drug eluting stented graft of claim 1 wherein said composite coating was applied to said stent by the steps of:
immersing said stent in a liquid dispersion of said composite;
removing said stent from said liquid dispersion of said composite; and,
drying said liquid dispersion of said composite that has remained on said stent,
whereby said composite coating is formed on said stent.

49. The drug eluting stented graft of claim 1 wherein said composite coating is formed by electron beam deposition.

50. The drug eluting stented graft of claim 1 wherein said tubular covering is adherent to said coat.

51. A method for the treatment of cardiovascular disease, comprising implanting the drug eluting stented graft of claim 1 in a patient in need of such treatment wherein said implantation is effective to ameliorate one or more of the symptoms of said cardiovascular disease.

52. An article of manufacture, comprising packaging material and the drug eluting stented graft of claim 1 contained within the packaging material, wherein said drug eluting stented graft is effective for implantation in a patient afflicted with cardiovascular disease, and the packaging material includes a label that indicates that said device is effective for said implantation.

53. In a tubular stented graft which is alternately deployable in a radially compact configuration having a first diameter and a radially expanded configuration having a second diameter, said stented graft comprising:
a stent comprising:
at least one member formed in a generally cylindrical shape having an outer surface and a hollow bore which extends longitudinally therethrough to define an inner surface;
said stent being initially radially collapsible to a diameter which is substantially equal to said first diameter of the stented graft, and subsequently radially expandable to a diameter which is substantially equal to said second diameter of the stented graft; and,
a plurality of lateral openings existing in said stent when said stent is at its radially expanded second diameter;
a continuous, tubular PTFE covering formed on said stent, said PTFE covering comprising:
a tubular inner base graft formed of expanded, sintered PTFE, said tubular base graft having an outer surface and an inner surface, said tubular base graft being deployed coaxially within the hollow bore of said stent such that the outer surface of the tubular base graft is in contact with the inner surface of the stent, and the inner surface of said tubular base graft thereby defining a luminal passageway through the stented graft; and,
a tubular outer layer formed of expanded, sintered PTFE tape which has a width of less than about 1 inch, said tape having been wound about the outer surface of said stent to create said tubular outer layer thereon, such that said stent is captured between said outer layer and said tubular base graft;
said tubular outer layer being attached to said tubular base graft, through said lateral openings in said stent, to thereby form an integrally stented, continuous PTFE tube which is alternately disposable in said radially compact configuration of said first diameter and said radially expanded configuration of said second diameter;
the improvement wherein said stent is coated with a coat comprising a composite of at least one polymer and at least one therapeutic substance to form a drug eluting stented graft.

54. The drug eluting stented graft of claim 53, wherein said at least one polymer is a biocompatible, pharmaceutically acceptable, bioerodible polymer.

55. The drug eluting stented graft of claim 53, wherein said at least one polymer is a polyester.

56. The drug eluting stented graft of claim 53, wherein said at least one therapeutic agent is selected from the group consisting of antiplatelet agents, anticoagulant agents, antimetabolic agents, antisense agents, vasoactive agents, nitric oxide releasing agents, anti-inflammatory agents, antiproliferative agents, pro-endothelial agents, anti-migratory agents, antimicrobial agents, selective gene delivery vectors, sirolimus, actinomycin-D and paclitaxel.

57. The drug eluting stented graft of claim 56, wherein said selective gene delivery vectors are Semliki Forest Virus (SMV) adapted to deliver restenosis preventing genes.

58. The drug eluting stented graft of claim 53, wherein said at least one polymer is a hydrophobic, bioerodible, copolymer comprising mers I and II according to the following formula: 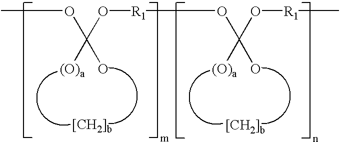
wherein:
R₁ is a member selected from the group consisting of alkylene of 1 to 10 carbons; alkenylene of 2 to 10 carbons; alkyleneoxy of 2 to 6 carbons; cycloalkylene of 3 to 7 carbons; cycloalkylene of 3 to 7 carbons substituted with a member selected from the group consisting of alkyl of 1 to 7 carbons, an alkoxy of 1 to 7 carbons, an alkylene of 1 to 10 carbons, and an alkenyl of 2 to 7 carbons; cycloalkenylene of 4 to 7 carbons; cycloalkenylene of 4 to 7 carbons substituted with an alkyl of 1 to 7 carbons, an alkoxy of 1 to 7 carbons, an alkylene of 1 to 10 carbons, and an alkenyl of 2 to 7 carbons; arylene; and arylene substituted with an alkyl of 1 to 7 carbons, an alkoxy of 1 to 7 carbons, an alkylene of 1 to 10 carbons, an alkenyl of 2 to 7 carbons; and wherein a is 0 to 1; b is 2 to 6; m is greater than 10; n is greater than 10; and at least one of R₁, a, and b in mer I is different than R₁, a, and b in mer II; and wherein:
said composite of at least one polymer and at least one therapeutic substance when in operation bioerodes and releases said at least one therapeutic substance at a rate selected from (1) a zero order rate, (2) a continuous rate, and (3) a variable rate, which rate is produced by preselecting said composite of at least one polymer and at least one therapeutic substance, and said elastomer to give the desired result.

59. The drug eluting stented graft of claim 53, wherein said at least one polymer is a hydrophobic, bioerodible, terpolymer comprising mers I, II, and III according to the following formula: [chemical structure]
wherein:
R₁ is a member selected from the group consisting of alkylene of 1 to 10 carbons; alkenylene of 2 to 10 carbons; alkyleneoxy of 2 to 6 carbons; cycloalkylene of 3 to 7 carbons; cycloalkylene of 3 to 7 carbons substituted with a member selected from the group consisting of alkyl of 1 to 7 carbons, alkoxy of 1 to 7 carbons, an alkylene of 1 to 10 carbons, and an alkenyl of 2 to 7 carbons; cycloalkenylene of 4 to 7 carbons; cycloalkenylene of 4 to 7 carbons substituted with an alkyl of 1 to 7 carbons, an alkoxy of 1 to 7 carbons, an alkylene of 1 to 10 carbons, and an alkenyl of 2 to 7 carbons; arylene; and arylene substituted with an alkyl of 1 to 7 carbons, an alkoxy of 1 to 7 carbons, an alkylene of 1 to 10 carbons, an alkenyl of 2 to 7 carbons; and wherein a is 0 to 1; b is 2 to 6; m is greater than 10; n is greater than 10; p is greater than 10; and at least one of R₁, a, and b in mers I, II and III is different than R₁, a, and b in mers I, II and III; and wherein:
said composite of at least one polymer and at least one therapeutic substance when in operation bioerodes and releases said at least one therapeutic substance at a rate selected from (1) a zero order rate, (2) a continuous rate, and (3) a variable rate, which rate is produced by preselecting said composite of said at least one polymer and said at least one therapeutic substance, and said elastomer to give the desired result.

60. The drug eluting stented graft of claim 53, wherein:
a multiplicity of microcapsules is dispersed within said at least one polymer, wherein said microcapsules have a wall formed of a drug release rate controlling material;
said at least one therapeutic substance is contained within said multiplicity of microcapsules; and,
said at least one polymer has the formula: 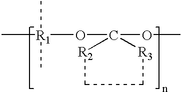
wherein R₁ is a member selected from the group of divalent, trivalent and tetravalent radicals consisting of alkylene of 1 to 10 carbons; alkenylene of 2 to 10 carbons; alkyleneoxy of 2 to 6 carbons; cycloalkylene of 3 to 7 carbons; cycloalkylene of 3 to 7 carbons substituted with an alkyl of 1 to 7 carbons, alkoxy of 1 to 7 carbons, an alkylene of 1 to 10 carbons, and an alkenyl of 2 to 7 carbons; cycloalkenylene of 4 to 7 carbons cycloalkenylene of 4 to 7 carbons substituted with an alkyl of 1 to 7 carbons, an alkoxy of 1 to 7 carbons, an alkylene of 1 to 10 carbons, and an alkenyl of 2 to 7 carbons; arylene; and arylene substituted with an alkyl of 1 to 7 carbons, an alkoxy of 1 to 7 carbons, and an alkenyl of 2 to 7 carbons; R₂ and R₃ are selected from the group consisting of alkyl of 1 to 7 carbons; alkenyl of 2 to 7 carbons; alkoxy of 1 to 7 carbons; alkenyloxy of 2 to 7 carbons; alkylene of 2 to 6 carbons; alkenylene of 3 to 6 carbons; alkyleneoxy of 2 to 6 carbons; alkenyleneoxy of 3 to 6 carbons; aryloxy; aralkyleneoxy of 8 to 12 carbons; aralkenyleneoxy of 8 to 12 carbons; oxa; OR₁O with R₁ as defined above; a heterocyclic ring of 5 to 8 carbon and oxygen atoms formed when R₂ and R₃ are taken together; a heterocyclic ring of 5 to 8 carbon and oxygen atoms substituted with an alkyl of 1 to 7 carbons, an alkoxy of 1 to 7 carbons and alkenyl of 2 to 7 carbons formed when R₂ and R₃ are taken together; a fused polycyclic ring of 8 to 12 carbon and oxygen atoms formed when R₂ and R₃ are taken together; a fused polycyclic ring of 8 to 12 carbon and oxygen atoms substituted with an alkyl of 1 to 7 carbons; an alkoxy of 1 to 7 carbons and an alkenyl of 2 to 7 carbons; and wherein at least one of said R₂ and R₃ is a member selected from the group consisting of alkoxy, alkenyloxy and OR₁O; R₂ and R₃ when taken together are a member selected from the group of heterocyclic and fused polycyclic rings having at least one oxygen atom in the ring; and wherein n is greater than 10;
so that, in operation, said polymer and said microcapsules bioerode at a controlled and continuous rate over a prolonged period of time, thereby releasing said at least one therapeutic substance at a controlled and continuous rate over a prolonged period of time.

61. The drug eluting stented graft of claim 53, wherein:
said coat further comprises at least a first layer and a second layer, wherein said first layer comprises said at least one therapeutic substance and at least a first polymer, and said second layer comprises said at least one therapeutic substance and at least a second polymer, wherein at least one of said first polymer and said second polymer are selected from the group consisting of polymers of the formula: 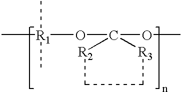
wherein R₁ is a member selected from the group of divalent, trivalent and tetravalent radicals consisting of alkylene of 1 to 10 carbons; alkenylene of 2 to 10 carbons; alkyleneoxy of 2 to 6 carbons; cycloalkylene of 3 to 7 carbons; cycloalkylene of 3 to 7 carbons substituted with an alkyl of 1 to 7 carbons, alkoxy of 1 to 7 carbons, alkylene of 1 to 10 carbons, and an alkenyl of 2 to 7 carbons; cycloalkenylene of 4 to 7 carbons; cycloalkenylene of 4 to 7 carbons substituted with an alkyl of 1 to 7 carbons, an alkoxy of 1 to 7 carbons, an alkylene of 1 to 10 carbons, and an alkenyl of 2 to 7 carbons; arylene; and arylene substituted with an alkyl of 1 to 7 carbons, an alkoxy of 1 to 7 carbons, and an alkenyl of 2 to 7 carbons; R₂ and R₃ are selected from the group consisting of alkyl of 1 to 7 carbons; alkenyl of 2 to 7 carbons; alkoxy of 1 to 7 carbons; alkenyloxy of 2 to 7 carbons; alkylene of 2 to 6 carbons; alkenylene of 3 to 6 carbons; alkyleneoxy of 2 to 6 carbons; alkenyleneoxy of 3 to 6 carbons; aryloxy; aralkyleneoxy of 8 to 12 carbons; aralkenyleneoxy of 8 to 12 carbons; oxa; OR₁O with R₁ as defined above; a heterocyclic ring of 5 to 8 carbon and oxygen atoms formed when R₂ and R₃ are taken together; a heterocyclic ring of 5 to 8 carbon and oxygen atoms substituted with an alkyl of 1 to 7 carbons; an alkoxy of 1 to 7 carbons and an alkenyl of 2 to 7 carbons formed when R₂ and R₃ are taken together; a fused polycyclic ring of 8 to 12 carbon and oxygen atoms formed when R₂ and R₃ are taken together; a fused polycyclic ring of 8 to 12 carbon and oxygen atoms substituted with an alkyl of 1 to 7 carbons, an alkoxy of 1 to 7 carbons and an alkenyl of 2 to 7 carbons; and wherein at least one of said R₂ and R₃ is a member selected from the group consisting of alkoxy, alkenyloxy and OR₁O; R₁ and R₃when taken together are a member selected from the group of heterocyclic and fused polycyclic rings having at least one oxygen atom in the ring; and wherein is greater than 10;
so that when in operation, said layers bioerode at a controlled and continuous rate over a prolonged period of time, thereby releasing said at least one therapeutic substance at a controlled and continuous rate over a prolonged period of time.

62. The drug eluting stented graft of claim 61, wherein said first polymer is a pharmaceutically acceptable biocompatible non-bioerodible polymer that sequesters an agent for brachytherapy.

63. The drug eluting stented graft of claim 62, wherein said agent for brachytherapy is selected from the group consisting of palladium-103 (¹⁰³Pd), ¹⁹²Ir, ³²P, ¹⁸⁸Re, and Sr/Y90 source trains.

64. The drug eluting stented graft of claim 53, wherein:
a multiplicity of discrete, closed cells exists within said at least one polymer, said cells having a wall formed and defined by said at least one polymer;
said at least one polymer has the formula: 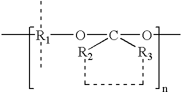
wherein R₁ is a member selected from the group of divalent, trivalent and tetravalent radicals consisting of alkylene of 1 to 10 carbons; alkenylene of 2 to 10 carbons; alkyleneoxy of 2 to 6 carbons; cycloalkylene of 3 to 7 carbons; cycloalkylene of 3 to 7 carbons substituted with an alkyl of 1 to 7 carbons, alkoxy of 1 to 7 carbons, an alkylene of 1 to 10 carbons, and an alkenyl of 2 to 7 carbons; cycloalkenylene of 4 to 7 carbons; cycloalkenylene of 4 to 7 carbons substituted with an alkyl of 1 to 7 carbons, an alkoxy of 1 to 7 carbons, an alkylene of 1 to 10 carbons, and an alkenyl of 2 to 7 carbons; arylene; and arylene substituted with an alkyl of 1 to 7 carbons, an alkoxy of 1 to 7 carbons, and an alkenyl of 2 to 7 carbons; R₂ and R₃ are selected from the group consisting of alkyl of 1 to 7 carbons; alkenyl of 2 to 7 carbons; alkoxy of 1 to 7 carbons; alkenyloxy of 2 to 7 carbons; alkylene of 2 to 6 carbons; alkenylene of 3 to 6 carbons; alkyleneoxy of 2 to 6 carbons; alkenyleneoxy of 3 to 6 carbons; aryloxy; aralkyleneoxy of 8 to 12 carbons; aralkenyleneoxy of 8 to 12 carbons; oxa; OR₁O with R₁ as defined above; a heterocyclic ring of 5 to 8 carbon and oxygen atoms formed when R₂ and R₃ are taken together; a heterocyclic ring of 5 to 8 carbon and oxygen atoms substituted with an alkyl of 1 to 7 carbons, an alkoxy of 1 to 7 carbons and an alkenyl of 2 to 7 carbons formed when R₂ and R₃ are taken together; a fused polycyclic ring of 8 to 12 carbon and oxygen atoms formed when R₂ and R₃ are taken together; a fused polycyclic ring of 8 to 12 carbon and oxygen atoms substituted with an alkyl of 1 to 7 carbons, an alkoxy of 1 to 7 carbons and an alkenyl of 2 to 7 carbons; and wherein at least one of said R₂ and R₃ is a member selected from the group consisting of alkoxy, alkenyloxy and OR₁O; R₂ and R₃when taken together are a member selected from the group of heterocyclic and fused polycyclic rings having at least one oxygen atom in the ring; and wherein n is greater than 10;
wherein said at least one therapeutic substance dissolved in a pharmaceutically acceptable carrier that is a solvent for said at least one therapeutic substance and a nonsolvent for said at least one polymer is contained within said multiplicity of discrete, closed cells;
so that, when in operation, said at least one polymer is capable of bioeroding at a controlled and continuous rate over a prolonged period of time, thereby releasing said at least one therapeutic substance at a controlled and continuous rate over a prolonged period of time.

65. The drug eluting stented graft of claim 53, wherein said stent comprises a plurality of elements, wherein each said element comprises an undulating linear shape formed into a generally cylindrical configuration having a cylinder axis generally aligned on the axis of said hollow bore, and wherein each said element is connected to an adjacent neighbor element by at least one linear connector.

66. The drug eluting stented graft of claim 65, wherein said plurality of elements comprises a spiral.

67. The drug eluting stented graft of claim 65, wherein at least one said connector is substantially circumferentially offset from an adjacent neighbor connector.

68. The drug eluting stented graft of claim 67, wherein said circumferentially offset connectors form a helical array.

69. The drug eluting stented graft of claim 65, wherein at least one said connector is not substantially circumferentially offset from an adjacent neighbor connector.

70. The drug eluting stented graft of claim 65, wherein said undulating linear shape is a generally zigzag shape comprising a plurality of zigs having tips and a plurality of zags having tips, wherein said tip of each said zig of each element and the nearest said tip of each said zig of an adjacent neighbor element generally lie in a plane passing through the axis of said hollow bore, and wherein said tip of at least one said zig of each element and at least one said nearest said tip of a zig of an adjacent neighbor are connected by one said linear connector.

71. The drug eluting stented graft of claim 65, wherein said undulating linear shape is a sinusoidal shape having a plurality of peaks and a plurality of valleys, wherein each said peak of each element and each said valley of an adjacent neighbor generally lie in a plane passing through the axis of said hollow bore, and wherein at least one said peak of each element and said valley of an adjacent neighbor lying generally in said plane are connected by one said linear connector.

72. The drug eluting stented graft of claim 65, wherein each said linear connector has a length dimension generally parallel to the axis of said hollow bore, and a width and depth dimension, and wherein said length dimension is greater than said width dimension and said length dimension is greater than said depth dimension.

73. The drug eluting stented graft of claim 72, wherein said length dimension is about 3 to 10 times greater than said width dimension, and said length dimension is about 3 to 10 times greater than said depth dimension.

74. The drug eluting stented graft of claim 53 wherein said PTFE is replaced by an elastomer selected from the group consisting of fluorinated ethylene propylene, polytetrafluoroethylene-perfluoroalkyl vinyl ether copolymer, polyvinyl chloride, polypropylene, polyethylene terephthalate, broad fluoride; and, other biocompatible plastics.

75. The drug eluting stented graft of claim 53 wherein said PTFE covering is formed of expanded, sintered PTFE tape, said tape having been wound about the outer surface of said stent to create said covering thereon.

76. The drug eluting stented graft of claim 53, wherein said PTFE is expanded polytetrafluoroethylene having fibrils.

77. The drug eluting stented graft of claim 76, wherein said fibrils measure up to about 300μ in length.

78. The drug eluting stented graft of claim 76, wherein said fibrils measure up to about 200μ in length.

79. The drug eluting stented graft of claim 76, wherein said fibrils measure up to about 100μ in length.

80. The drug eluting stented graft of claim 76, wherein said fibrils measure up to about 50μ in length.

81. The drug eluting stented graft of claim 76, wherein said fibrils measure up to about 5μ in length.

82. The drug eluting stented graft of claim 75 wherein said tape has a width of less than about 1 inch (2.54 cm.).

83. The drug eluting stented graft of claim 75 wherein said tape has a thickness of less than 0.015 inch (0.038 cm.) and wherein said tape is wound about said stent in overlapping fashion, such that said elastomer covering comprises 1 to 10 layers of said tape.

84. The drug eluting stented graft of claim 75 wherein said tape is helically wrapped about said stent.

85. The drug eluting stented graft of claim 75 wherein said tape has a width of 0.5 inches (1.27 cm), and wherein said tape is helically wrapped such that 6-8 revolutions of tape are applied per longitudinal inch (2.54 cm.) of said drug eluting stented graft.

86. The drug eluting stented graft of claim 75 wherein said tape is helically wrapped alternately in a first direction and then in the opposite direction.

87. The drug eluting stented graft of claim 86 further comprising 8 layers of said tape.

88. The drug eluting stented graft of claim 53 wherein said stent is a self-expanding stent.

89. The drug eluting stented graft of claim 88, wherein said self-expanding stent comprises a shape memory alloy that can alternately exist in a first and a second crystalline state, wherein said stent assumes a radially expanded configuration when said shape memory alloy is in said first crystalline state, and a radially compact configuration when said shape memory alloy is in said second crystalline state.

90. The drug eluting stented graft of claim 53 wherein said stent is a pressure-expandable stent.

91. The drug eluting stented graft of claim 88 wherein said stent is formed of a metal alloy comprising at least two elements selected from the group consisting of iron, cobalt, chromium, nickel, titanium, niobium, and molybdenum.

92. The drug eluting stented graft of claim 89 wherein said shape memory alloy comprises at least about 51% to about 59% nickel and the remainder comprising titanium.

93. The drug eluting stented graft of claim 89 wherein said shape memory alloy comprises about 0.25% chromium, at least about 51% to about 59% nickel, and the remainder comprising titanium.

94. The drug eluting stented graft of claim 53 wherein said covering has a thickness of less than 0.1 inch (0.25 cm.).

95. The drug eluting stented graft of claim 75 wherein said PTFE tape has a thickness of less than 0.015 inches (0.038 cm.), said tape being wrapped about said stent in overlapping fashion so as to form said covering.

96. The drug eluting stented graft of claim 75 wherein said PTFE tape has a density of less than 1.6 g/cc.

97. The drug eluting stented graft of claim 75 wherein said covering has a thickness of less than 0.1 inch (0.25 cm.) and the PTFE tape has a density of less than 1.6 g/cc.

98. The drug eluting stented graft of claim 53 wherein said coat was applied to said stent by the steps of:
immersing said stent in a liquid polymer dispersion;
removing said stent from said liquid polymer dispersion; and,
drying said liquid polymer dispersion that has remained on said stent,
whereby said coat is formed on said stent.

99. The drug eluting stented graft of claim 53 wherein said coat is formed by electron beam deposition.

100. The drug eluting stented graft of claim 53 wherein said tubular covering is adherent to said coat.

101. A method for the treatment of cardiovascular disease, comprising implanting the drug eluting stented graft of claim 53 in a patient in need of such treatment wherein said implantation is effective to ameliorate one or more of the symptoms of said cardiovascular disease.

102. An article of manufacture, comprising packaging material and the drug eluting stented graft of claim 53 contained within the packaging material, wherein said drug eluting stented graft is effective for implantation in a patient afflicted with cardiovascular disease, and the packaging material includes a label that indicates that said device is effective for said implantation.
